(19) United States
(12) Patent Application Publication
(10) Pub. No.: US 2025/0101432 A1
(43) Pub. Date: Mar. 27, 2025

(54) DEGRADATION OF RNA BY THE LYSOSOMAL PATHWAY

(71) Applicants: Micah Belew, Somerville, MA (US); UNIVERSITY OF MASSACHUSETTS, Boston, MA (US)

(72) Inventors: Micah Belew, Somerville, MA (US); Dilara O. Halim, Worcester, MA (US); Fen-Biao Gao, Chestnut Hill, MA (US); Alejandro Vasquez Rifo, Worcester, MA (US)

(21) Appl. No.: 18/799,165

(22) Filed: Aug. 9, 2024

Related U.S. Application Data
(60) Provisional application No. 63/532,208, filed on Aug. 11, 2023.

(51) Int. Cl. C12N 15/113; A61P 25/28; C12N 15/86

(52) U.S. Cl. CPC C12N 15/1137; A61P 25/28; C12N 15/86; C12N 2310/11; C12N 2310/315; C12N 2310/321; C12N 2310/322; C12N 2310/3341; C12N 2310/351; C12N 2750/14143; C12Y 115/01001; C12Y 304/21061

(57) ABSTRACT

The present disclosure provides oligonucleotides, methods, and compositions for degrading RNA via the lysosomal pathway. Also contemplated are oligonucleotides, methods, and compositions for the treatment, prevention, or amelioration of diseases, disorders, and conditions associated with EXOC2, Ku80, and Task1 in a subject in need thereof.

### CROSS REFERENCE TO RELATED APPLICATION

This application claims the benefit of U.S. Provisional Patent Application Ser. No. 63/532,208, filed Aug. 11, 2023. The entire content of the above-referenced patent application is incorporated by reference in its entirety herein.

### SEQUENCE LISTING

The instant application contains a Sequence Listing which has been submitted electronically in XML format and is hereby incorporated by reference in its entirety. Said XML file, created on Dec. 10, 2024, is named 755176_UM9-307_ST26.xml and is 137,916 bytes in size.

### BACKGROUND

RNA knockdown-based approaches have emerged as powerful tools to manipulate gene expression and elucidate gene functions (Fire et al., 1998; Hannon, 2002). Utilizing small RNA molecules, such as small interfering RNAs (siRNAs) and short hairpin RNAs (shRNAs), these methods offer the potential to selectively silence gene expression with high precision and specificity (Elbashir et al., 2001; Brummelkamp et al., 2002). Despite their promise, these approaches have faced challenges, particularly in terms of efficacy and target specificity (Jackson & Linsley, 2010; Boudreau et al., 2011).

One of the critical limitations encountered in RNA knockdown methodologies is related to the intracellular fate of the RNA molecules. Cellular uptake and subsequent processing are intricate processes that can profoundly influence the knockdown efficiency (Juliano, 2016). Moreover, suboptimal endosomal escape and premature degradation in the lysosomal compartments have been identified as major obstacles that hamper the effectiveness of these approaches (Gilleron et al., 2013; Daka et al., 2020).

Accordingly, there exists a need for more efficient RNA knockdown methodologies.

### SUMMARY

The present disclosure provides oligonucleotides, methods, and compositions for degrading RNA via the lysosomal pathway.

In one aspect, the disclosure provides an oligonucleotide comprising a 5′ end, a 3′ end, and complementarity to a target polynucleotide, wherein the oligonucleotide comprises a poly-G sequence linked to the 5′ end and/or 3′ end of the oligonucleotide, and wherein the poly-G sequence lacks complementarity to the target polynucleotide.

In some embodiments, the poly-G sequence comprises 2-30 G nucleotides.

In some embodiments, the poly-G sequence is 2, 3, 4, 5, 6, 7, 8, 9, 10, 11, 12, 13, 14, or 15 G nucleotides (SEQ ID NO: 57). In some embodiments, the poly-G sequence comprises or consists of 5 G nucleotides. In some embodiments, the poly-G sequence comprises or consists of 6 G nucleotides. In some embodiments, the poly-G sequence comprises or consists of 7 G nucleotides. In some embodiments, the poly-G sequence comprises or consists of 8 G nucleotides. In some embodiments, the poly-G sequence comprises or consists of 9 G nucleotides. In some embodiments, the poly-G sequence comprises or consists of 10 G nucleotides. In some embodiments, the poly-G sequence comprises or consists of 11 G nucleotides. In some embodiments, the poly-G sequence comprises or consists of 12 G nucleotides. In some embodiments, the poly-G sequence comprises or consists of 13 G nucleotides. In some embodiments, the poly-G sequence comprises or consists of 14 G nucleotides. In some embodiments, the poly-G sequence comprises or consists of 15 G nucleotides.

In some embodiments, the poly-G sequence is linked to the 3′ end of an oligonucleotide of the disclosure. In other embodiments, the poly-G sequence is linked to the 5′ end of an oligonucleotide of the disclosure. In other embodiments, the poly-G sequence is linked to the 5′ end and the 3′ end of an oligonucleotide of the disclosure.

In some embodiments, the poly-G sequence is single stranded.

In some embodiments, the poly-G sequence binds to a protein on the surface of a lysosome. In some embodiments, the poly-G sequence binds to a lysosome-associated membrane glycoprotein (LAMP). In some embodiments, the LAMP is LAMP2C. In some embodiments, the poly-G sequence comprises G nucleotides that are consecutive. In some embodiments, the poly-G sequence comprises or consists of 5-15 consecutive G nucleotides. In some embodiments, the poly-G sequence comprises 1 or more non-G nucleotides (e.g., A, T, U, or C) within the poly-G sequence. In some embodiments, the oligonucleotide comprises two or more poly-G sequences with 1 or more non-G nucleotides (e.g., A, T, U, or C) between the two or more poly-G sequences.

In some embodiments, the poly-G sequence comprises G nucleotides that are consecutive, not consecutive, or a combination thereof.

In some embodiments, the poly-G sequence comprises or consists of (dG)(dG)(dG)(dG)(dG). In some embodiments, the poly-G sequence comprises or consists of (dG)(dG)(dG)(dG)(dG)(dG). In some embodiments, the poly-G sequence comprises or consists of (dG)(dG)(dG)(dG)(dG)(dG)(dG). In some embodiments, the poly-G sequence comprises or consists of (dG)(dG)(dG)(dG)(dG)(dG)(dG)(dG). In some embodiments, the poly-G sequence comprises or consists of (dG)(dG)(dG)(dG)(dG)(dG)(dG)(dG)(dG). In some embodiments, the poly-G sequence comprises or consists of (dG)(dG)(dG)(dG)(dG)(dG)(dG)(dG)(dG)(dG)(SEQ ID NO: 56). In some embodiments, the poly-G sequence comprises or consists of (dG)(dG)(dG)(dN)(dG)(dG)(dG)(dN)(dG), wherein each dN individually corresponds to a deoxyribonucleotide of dA, dT, or dC. In some embodiments, the poly-G sequence comprises or consists of (dG)(dG)(dG)(dN)(dG)(dG)(dG)(dN)(dG)(dG)(SEQ ID NO: 58), wherein each dN individually corresponds to a deoxyribonucleotide of dA, dT, or dC. In some embodiments, the poly-G sequence comprises or consists of (dG)(dG)(dG)(dN)(dG)(dG)(dG)(dN)(dG)(dG) (SEQ ID NO: 58), wherein each dN individually corresponds to a deoxyribonucleotide of dA, dT, or dC.

In some embodiments, the oligonucleotide is about 10 nucleotides to about 35 nucleotides in length.

In some embodiments, the oligonucleotide is 10, 11, 12, 13, 14, 15, 16, 17, 18, 19, 20, 21, or 22 nucleotides in length.

In some embodiments, the oligonucleotide and/or poly-G sequence comprise one or more modified nucleotides.

In some embodiments, the one or more modified nucleotides each independently comprise a modification of a ribose group, a phosphate group, a nucleobase, or a combination thereof.

In some embodiments, each modification of the ribose group comprises 2′-O-methyl, 2′-fluoro, 2′-deoxy, 2′-O-(2-methoxyethyl)(MOE), 2′-O-alkyl, 2′-O-alkoxy, 2′-O-alkylamino, 2′-NH2, a constrained nucleotide, or a combination thereof.

In some embodiments, the constrained nucleotide comprises a locked nucleic acid (LNA), an ethyl-constrained nucleotide, a 2′-(S)-constrained ethyl(S-cEt) nucleotide, a constrained MOE, a 2′-0, 4′-C-aminomethylene bridged nucleic acid (2′,4′-BNANC), an alpha-L-locked nucleic acid, a tricyclo-DNA, or a combination thereof.

In some embodiments, the modification of the ribose group comprises a 2′-O-(2-methoxyethyl)(MOE) modification.

In some embodiments, the nucleotides at the position 1, 2, 3, 4, 5, 6, 7, 8, 9, and/or 10 from the 5′ end and/or 3′ end of the oligonucleotide comprise a 2′-O-(2-methoxyethyl)(MOE) modification.

In some embodiments, every nucleotides of the oligonucleotide and/or poly-G sequence comprise a 2′-O-(2-methoxyethyl)(MOE) modification.

In some embodiments, the modification of the ribose group comprises a tricyclo-DNA modification.

In some embodiments, every nucleotide of the oligonucleotide and/or poly-G sequence comprise a tricyclo-DNA modification.

In some embodiments, the modification of the ribose group comprises a 2′-deoxy modification.

In some embodiments, each modification of the phosphate group comprises a phosphorothioate, a phosphonoacetate (PACE), a thiophosphonoacetate (thioPACE), an amide, a triazole, a phosphonate, a phosphotriester, or a combination thereof.

In some embodiments, the modification of the phosphate group is phosphorothioate.

In some embodiments, every nucleotide of the oligonucleotide and/or the poly-G sequence comprise a phosphorothioate.

In some embodiments, the oligonucleotide and/or poly-G sequence comprise at least one phosphodiester intemucleotide linkage.

In some embodiments, every intemucleotide linkage in the oligonucleotide and/or poly-G sequence is a phosphodiester internucleotide linkage.

In some embodiments, each modification of the nucleobase comprises a 2-thiouridine, a 4-thiouridine, a N6-methyladenosine, a pseudouridine, a 2,6-diaminopurine, inosine, a thymidine, a 5-methylcytosine, a 5-substituted pyrimidine, an isoguanine, an isocytosine, a halogenated aromatic group, or a combination thereof.

In some embodiments, the modification of the nucleobase group comprises a 5-methylcytosine modification.

In some embodiments, the oligonucleotide comprises a mixture of modified nucleotides.

In some embodiments, a functional moiety is linked to the 5′ end or 3′ end of the oligonucleotide.

In some embodiments, the functional moiety comprises an N-acetylgalactosamine (GalNAc) moiety and/or a hydrophobic moiety.

In some embodiments, the hydrophobic moiety is selected from the group consisting of fatty acids, steroids, secosteroids, lipids, gangliosides, nucleoside analogs, endocannabinoids, vitamins, and a mixture thereof.

In some embodiments, the steroid selected from the group consisting of cholesterol and Lithocholic acid (LCA).

In some embodiments, the fatty acid selected from the group consisting of Eicosapentaenoic acid (EPA), Docosahexaenoic acid (DHA) and Docosanoic acid (DCA).

In some embodiments, the oligonucleotide comprises the formula:

A-B-C, wherein:
A comprises from about 0 to about 8 modified nucleotides;
B comprises from about 6 to about 18 deoxyribonucleic acid (DNA) nucleotides and/or DNA-like nucleotides; and
C comprises from about 0 to about 8 modified nucleotides;

and wherein the overall length of the antisense oligonucleotide is about 10 to about 30 nucleotides.

In some embodiments, A comprises from about 2 to about 6 modified nucleotides, B comprises from about 6 to about 12 DNA nucleotides and/or DNA-like nucleotides, and C comprises from about 2 to about 6 modified nucleotides.

In some embodiments, A comprises about 5 modified nucleotides, B comprises about 10 DNA nucleotides and/or DNA-like nucleotides, and C comprises about 5 modified nucleotides.

In some embodiments, A comprises from about 2 to about 6 2′-O-(2-methoxyethyl) (MOE) modified nucleotides, B comprises from about 6 to about 12 DNA-like nucleotides, and C comprises from about 2 to about 6 2′-O-(2-methoxyethyl)(MOE) modified nucleotides.

In some embodiments, A comprises about 5 2′-O-(2-methoxyethyl)(MOE) modified nucleotides, B comprises about 10 DNA-like nucleotides, and C comprises about 5 2′-O-(2-methoxyethyl)(MOE) modified nucleotides.

In some embodiments, the oligonucleotide comprises a nucleic acid sequence with at least 90% sequence identity of any one of the nucleic acid sequence of Table 1.

In some embodiments, the oligonucleotide comprises a sequence modification pattern of XsXsXsXsXsXsXsXsXsXsXsXsXsXsXsXsXsXsXsX, wherein
s represents a phosphorothioate internucleoside linkage;
X comprises an adenosine, a guanosine, a cytidine, a thymine, or a uracil, wherein X comprises a 2′-O-(2-methoxyethyl) modification; and
X comprises an adenosine, a guanosine, a cytidine, a thymine, or a uracil, wherein X comprises a 2′-deoxy modification.

In some embodiments, the oligonucleotide comprises a sequence modification pattern of XsXsXsXsXsXsXsXsXsXsXsXsXsXsXsXsXsX, wherein
s represents a phosphorothioate internucleoside linkage; and
X comprises an adenosine, a guanosine, a cytidine, a thymine, or a uracil, wherein X comprises a 2′-O-(2-methoxyethyl) modification.

In some embodiments, the oligonucleotide comprises a sequence modification pattern of XsXsXsXsXsXsXsXsXsXsXsXsXsXsXsXsXsXsXsX, wherein
s represents a phosphorothioate internucleoside linkage; and
X comprises an adenosine, a guanosine, a cytidine, a thymine, or a uracil, wherein X comprises a 2′-O-(2-methoxyethyl) modification.

In some embodiments, the target polynucleotide is mammalian or viral mRNA. In some embodiments, the target polynucleotide is an intronic or exonic region of the mRNA.

In some embodiments, the target is selected from the group consisting of a EXOC2 gene, a Ku80 gene, and a Task1 gene.

In some embodiments, the oligonucleotide inhibits the expression of the EXOC2 gene, the Ku80 gene, or the Task1 gene by at least about 50%.

In some embodiments, the target is SOD1.

In some embodiments, the oligonucleotide of comprises the sequence (eC) #(eA)(eG) #(eG)(eA) #(dT) #(dA) #(d5C) #(dA) #(dT) #(dT) #(dT) #(d5C) #(dT) #(dA) #(eC)(e A) #(eG)(eC) #(eU)(polyG)(SEQ ID NO: 48), wherein (#) indicates phosphorothioate linkage, (e) indicates a 2′MOE modification, (d) indicates a deoxyribonucleotide, (dN) indicates a deoxyribonucleotide of A, T, or C, (d5C) indicates 5-methyl cytosine, and (polyG) indicates 2-30 G nucleotides.

In some embodiments, the oligonucleotide of comprises the sequence (eC) #(eA)(eG) #(eG)(eA) #(dT) #(dA) #(d5C) #(dA) #(dT) #(dT) #(dT) #(d5C) #(dT) #(dA) #(eC)(e A) #(eG)(eC) #(eU)(dG)ₓ(SEQ ID NO: 49), wherein (#) indicates phosphorothioate linkage, (e) indicates a 2′MOE modification, (d) indicates a deoxyribonucleotide, (d5C) indicates 5-methyl cytosine, and x indicates an integer between 5-10.

In some embodiments, the oligonucleotide of comprises the sequence (eC) #(eA)(eG) #(eG)(eA) #(dT) #(dA) #(d5C) #(dA) #(dT) #(dT) #(dT) #(d5C) #(dT) #(dA) #(eC)(e A) #(eG)(eC) #(eU)(dGdGdGdN)ₓ(SEQ ID NO: 50), wherein (#) indicates phosphorothioate linkage, (e) indicates a 2′MOE modification, (d) indicates a deoxyribonucleotide, (d5C) indicates 5-methyl cytosine, (dN) indicates a deoxyribonucleotide of A, T, or C, and x indicates an integer between 2-10.

In some embodiments, the target is PCSK9.

In some embodiments, the oligonucleotide is selected from the group consisting of an antisense oligonucleotide (ASO), a gapmer, a siRNA, a miRNA, a shRNA, a CRISPR guide, a DNA, an antisense mixmer, a miRNA inhibitor, a splice-switching oligonucleotide (SSO), a phosphorodiamidate morpholino oligomer (PMO), and a peptide nucleic acid (PNA).

In some embodiments, the oligonucleotide is a double-stranded RNA (dsRNA).

In some embodiments, the dsRNA comprises an antisense strand complementary to a target polynucleotide. In some embodiments, the antisense strand is about 10-35 nucleotides in length. In some embodiments, the antisense strand is 18, 19, 20, 21, 22, or 23 nucleotides in length. In some embodiments, the dsRNA comprises a sense strand that is complementary to at least a portion of the antisense strand. In some embodiments, the sense strand is about 10-35 nucleotides in length. In some embodiments, the sense strand is 13, 14, 15, 16, 17, 18, 19, 20, 21, 22, or 23 nucleotides in length.

In some embodiments, the poly-G sequence is linked to the 5′ end and/or 3′ end of the antisense strand and/or sense strand.

In some embodiments, the target is PCSK9.

In some embodiments, the dsRNA comprises an antisense strand of V(mA) #(fC) #(mA)(fA)(fA)(fA)(mG)(fC)(mA)(fA)(mA)(mA)(mC)(fA)(mG)(fG)(mU)(fC)(m U)(mA)(mG) #(mA) #(mA)(SEQ ID NO: 40) and a sense strand of (mC) #(mU) #(mA)(mG)(mA)(mC)(fC)(mU)(fG)(mU)(dT)(mU)(mU)(mG)(mC)(mU)(mU)(m U)(mU)(mG)(mU)GalNac(SEQ ID NO: 39), wherein (#) indicates a phosphorothioate linkage, (mN) indicates a 2′-OMe modification, (fN) indicates a 2′-Fluoro modification, V indicates 5′-Vinyl phosphate, and GalNac indicates a N-acetylgalactosamine (GalNAc) conjugate.

In some embodiments, the dsRNA comprises an antisense strand of V(mA) #(fC) #(mA)(fA)(fA)(fA)(mG)(fC)(mA)(fA)(mA)(mA)(mC)(fA)(mG)(fG)(mU)(fC)(m U)(mA)(mG) #(mA) #(mA)(polyG)(SEQ ID NO: 51), wherein (#) indicates a phosphorothioate linkage, (mN) indicates a 2′-OMe modification, (fN) indicates a 2′-Fluoro modification, V indicates 5′-Vinyl phosphate, and (polyG) indicates 2-30 G nucleotides.

In some embodiments, the dsRNA comprises an antisense strand of V(mA) #(fC) #(mA)(fA)(fA)(fA)(mG)(fC)(mA)(fA)(mA)(mA)(mC)(fA)(mG)(fG)(mU)(fC)(m U)(mA)(mG) #(mA) #(mA)(dG)ₓ(SEQ ID NO: 52), wherein (#) indicates a phosphorothioate linkage, (mN) indicates a 2′-OMe modification, (fN) indicates a 2′-Fluoro modification, V indicates 5′-Vinyl phosphate, and x indicates an integer between 5-10.

In another aspect, the disclosure provides a pharmaceutical composition for inhibiting the expression of a gene in an organism comprising the oligonucleotide or the dsRNA, and a pharmaceutically acceptable carrier.

In some embodiments, the gene is selected from the group consisting of a EXOC2 gene, a Ku80 gene, and a Task1 gene.

In some embodiments, the oligonucleotide or the dsRNA inhibit the expression of the EXOC2 gene, the Ku80 gene, or the Task1 gene by at least about 50%.

In some embodiments, the oligonucleotide or the dsRNA inhibit the expression of the EXOC2 gene, the Ku80 gene, or the Task1 gene by at least about 80%.

In another aspect, the disclosure provides a vector comprising a regulatory sequence operably linked to a nucleotide sequence that encodes the oligonucleotide or the dsRNA.

In some embodiments, the oligonucleotide or the dsRNA inhibit the expression of a gene by at least 30%

In some embodiments, the oligonucleotide or the dsRNA inhibit the expression of a gene by at least about 50%.

In some embodiments, the oligonucleotide or the dsRNA inhibit the expression of a gene by at least about 80%.

In some embodiments, the gene is selected from the group consisting of a EXOC2 gene, a Ku80 gene, and a Task1 gene.

In another aspect, the disclosure provides a recombinant adeno-associated virus (rAAV) comprising the vector and an AAV capsid.

In another aspect, the disclosure provides a cell comprising the vector or the rAAV.

In another aspect, the disclosure provides a method for inhibiting expression of a gene in a cell, the method comprising:
(a) introducing into the cell the oligonucleotide, the dsRNA, the vector, or the rAAV; and
(b) maintaining the cell produced in step (a) for a time sufficient to obtain degradation of the mRNA transcript of the gene, thereby inhibiting expression of the gene in the cell.

In some embodiments, the poly-G sequence directs the degradation from a lysosome.

In some embodiments, the gene is selected from the group consisting of a EXOC2 gene, a Ku80 gene, and a Task1 gene.

In some embodiments, the oligonucleotide, the dsRNA, the vector, or the rAAV inhibit the expression of the EXOC2 gene, the Ku80 gene, or the Task1 gene by at least about 50%.

In some embodiments, the oligonucleotide, the dsRNA, the vector, or the rAAV inhibit the expression of the EXOC2 gene, the Ku80 gene, or the Task1 gene by at least about 80%.

In some embodiments, the oligonucleotide, the dsRNA, the vector, or the rAAV is administered by intravenous (IV) injection, subcutaneous (SQ) injection, or a combination thereof.

In another aspect, the disclosure provides a method of treating or managing a disease associated with a gene comprising administering to a patient in need of such treatment a therapeutically effective amount of the oligonucleotide, the dsRNA, the vector, or the rAAV.

In some embodiments, the gene is selected from the group consisting of a EXOC2 gene, a Ku80 gene, and a Task1 gene.

In some embodiments, the oligonucleotide, the dsRNA, the vector, or the rAAV inhibit the expression of the EXOC2 gene, the Ku80 gene, or the Task1 gene by at least about 50%.

In some embodiments, the oligonucleotide, the dsRNA, the vector, or the rAAV inhibit the expression of the EXOC2 gene, the Ku80 gene, or the Task1 gene by at least about 80%.

In another aspect, the disclosure provides a method for degrading a target polynucleotide in a lysosome of a cell, the method comprising introducing the oligonucleotide described herein, the dsRNA described herein, the vector described herein, or the rAAV described herein, into the cell, and maintaining the cell for time sufficient to degrade the target polynucleotide in the lysosome of the cell.

In some embodiments, the method for degrading the target polynucleotide in the lysosome of the cell occurs in vivo, ex vivo, or in vitro.

In some embodiments, the target polynucleotide is mammalian or viral mRNA. In some embodiments, the target polynucleotide is an intronic or exonic region of the mRNA.

In some embodiments, the target is selected from the group consisting of a EXOC2 gene, a Ku80 gene, and a Task1 gene.

In some embodiments, the oligonucleotide inhibits the expression of the EXOC2 gene, the Ku80 gene, or the Task1 gene by at least about 50%.

In another aspect, the disclosure provides a method of treating or managing amyotrophic lateral sclerosis (ALS) in a patient, comprising administering to the patient a therapeutically effective amount of an oligonucleotide with complementarity to SOD1 described herein.
In another aspect, the disclosure provides a method of treating or managing primary hyperlipidemia in a patient, comprising administering to the patient a therapeutically effective amount of the dsRNA targeting PCSK9 described herein.

In some embodiments, the primary hyperlipidemia is heterozygous familial hypercholesterolemia (HeFH).

In another aspect, the disclosure provides a method of reducing low density lipoprotein cholesterol (LDL-C) in a patient, comprising administering to the patient a therapeutically effective amount of the dsRNA targeting PCSK9 described herein.

### BRIEF DESCRIPTION OF THE DRAWINGS

The foregoing and other features and advantages of the present disclosure will be more fully understood from the following detailed description of illustrative embodiments taken in conjunction with the accompanying drawings. The patent or application file contains at least one drawing executed in color. Copies of this patent or patent application publication with color drawing(s) will be provided by the Office upon request and payment of the necessary fee.

FIG. 1 depicts a schematic of the RNase H and lysosome based knockdown approaches to knockdown a target RNA.

FIG. 2 depicts a schematic of an exemplary Lyso-ASO that can target lysosome. The Gapmer ASO contains 5′/3′ terminal 2′MOE modifications with internal DNA nucleotides. Each intemucleotide linkage in the ASO is a phosphorothioate modification. The lyso sequence is unmodified.

FIG. 3 depicts relative EXOC2 mRNA level as measured by qRT-PCT analysis for gapmers with and without poly-G, poly-A, or poly-C ligand. N=7-9. Values are mean±S.E.M. *p<0.05; **p<0.01; ***p<0.001; **** p<0.0001, by two-tailed Student's t-test.

FIG. 4 (SEQ ID NOs: 56 and 59) depicts relative EXOC2 mRNA level as measured by qRT-PCT analysis for gapmers with and without poly-G ligand of different lengths. N=6-12. Values are mean±S.E.M. *p<0.05; **p<0.01; ***p<0.001; **** p<0.0001, by two-tailed Student's t-test.

FIG. 5 depicts relative EXOC2 mRNA level as measured by qRT-PCT analysis for EXOC2 349. N=3, independent differentiations. Values are mean±S.E.M. *p<0.05; **p<0.01; ***p<0.001; **** p<0.0001, by two-tailed Student's t-test.

FIG. 6 depicts relative EXOC2 mRNA level as measured by qRT-PCT analysis for EXOC2 3933. N=6. Values are mean±S.E.M. *p<0.05; **p<0.01; ***p<0.001; **** p <0.0001, by two-tailed Student's t-test.

FIG. 7 depicts a schematic of the BAFILOMYCIN A1 (Baf A1) inhibition of lysosomal V-ATPase and thus of lysosomal function.

FIG. 8 depicts relative EXOC2 mRNA level as measured by qRT-PCT analysis for EXOC2 3933 with inhibited and un-inhibited lysosome. N=6. Values are mean±S.E.M. *p <0.05; **p<0.01; ***p<0.001; **** p<0.0001, by two-tailed Student's t-test.

FIG. 9 depicts a schematic of the RNase H and lysosome based knockdown approaches to knockdown a target RNA using a sterically blocked ASO.

FIG. 10 depicts relative EXOC2 mRNA level as measured by qRT-PCT analysis for EXOC2 3933 that is sterically blocked and not blocked. N=4-6. Values are mean±S.E.M. *p <0.05; **p<0.01; ***p<0.001; **** p<0.0001, by two-tailed Student's t-test.

FIG. 11 depicts relative Ku80 mRNA level as measured by qRT-PCT analysis for Ku80 624 and 2802. N=6. Values are mean±S.E.M. *p<0.05; **p<0.01; ***p<0.001; **** p <0.0001, by two-tailed Student's t-test.

FIG. 12 depicts relative TASK1 mRNA level as measured by qRT-PCT analysis for TASK1 622 and 5968. N=6. Values are mean±S.E.M. *p<0.05; **p<0.01; ***p<0.001; **** p<0.0001, by two-tailed Student's t-test.

FIG. 13 depicts relative SOD1 mRNA level as measured by qRT-PCT analysis in SH-SY5Y cells treated with 25 or 100 nM Nontargeting control (NTC), Tofersen, or Tofersen-Lamp ASOs for 24 hrs (n=3). Values are mean±S.E.M. *p<0.05; **p<0.01; ***p<0.001; **** p<0.0001, by one-tailed t-test. FIG. 14 depicts relative PCSK9 mRNA level as measured by qRT-PCT analysis in A549 cells treated with 50 nM Inclisiran or Inclisiran-Lamp for 48 hrs (n=4). Values are mean±S.E.M. *p<0.05; **p<0.01; ***p<0.001; **** p<0.0001, by one-tailed t-test.

FIG. 15 depicts relative SOD1 mRNA level as measured by qRT-PCT analysis in SH-SY5Y cells treated with Vehicle (Mock), 30 nM Tofersen-Lamp, or Tofersen-Lamp derivative ASOs for 30 hrs (n=3). Values are mean±S.E.M. *p<0.05; **p<0.01; ***p<0.001; **** p<0.0001, by one- or two-tailed t-test.

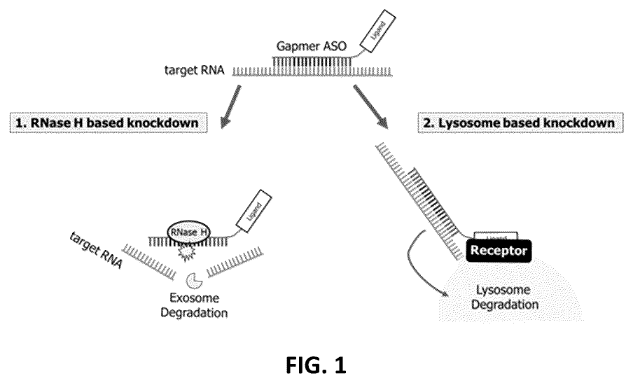

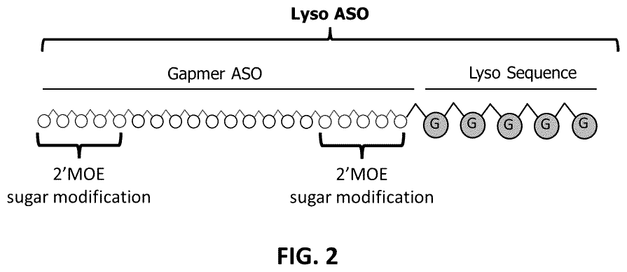

### DETAILED DESCRIPTION

The present disclosure provides oligonucleotides, methods, and compositions for degrading a target polynucleotide (e.g., an RNA) using the lysosomal pathway. The disclosure described herein is largely based on the finding that antisense compounds conjugated with a lysosome ligand (e.g., a poly-G sequence) facilitate the degradation of a target polynucleotide via the lysosomal pathway. Without being bound to any specific theory, the lysosome ligand from the antisense compound interacts with the LAMP-2C protein of the lysosome and triggers the uptake and degradation by the lysosome of the target polynucleotide attached to the antisense compound (e.g., an antisense oligonucleotide).

It is to be understood that the methods described in this disclosure are not limited to particular methods and experimental conditions disclosed herein as such methods and conditions can vary. It is also to be understood that the terminology used herein is for the purpose of describing particular embodiments only, and is not intended to be limiting.

Furthermore, the experiments described herein, unless otherwise indicated, can use conventional molecular and cellular biological and immunological techniques within the skill of the art. Such techniques are well known to the skilled worker, and are explained fully in the literature. See, e.g., Ausubel, et al., ed., Current Protocols in Molecular Biology, John Wiley & Sons, Inc., NY, N.Y. (1987-2008), including all supplements, Molecular Cloning: A Laboratory Manual (Fourth Edition) by M R Green and J. Sambrook and Harlow et al., Antibodies: A Laboratory Manual, Chapter 14, Cold Spring Harbor Laboratory, Cold Spring Harbor (2013, 2^{nd} edition).

Unless otherwise defined herein, scientific and technical terms used herein have the meanings that are commonly understood by those of ordinary skill in the art. In the event of any latent ambiguity, definitions provided herein take precedent over any dictionary or extrinsic definition. Unless otherwise required by context, singular terms shall include pluralities and plural terms shall include the singular. The use of “or” means “and/or” unless stated otherwise. The use of the term “including,” as well as other forms, such as “includes” and “included,” is not limiting.

Generally, nomenclature used in connection with cell and tissue culture, molecular biology, immunology, microbiology, genetics and protein and nucleic acid chemistry and hybridization described herein are those well-known and commonly used in the art. The nomenclature used in connection with, and the laboratory procedures and techniques of, analytical chemistry, synthetic organic chemistry, and medicinal and pharmaceutical chemistry described herein are those well-known and commonly used in the art.

So that the disclosure can be more readily understood, certain terms are first defined.

The term “nucleoside” refers to a molecule having a purine or pyrimidine base covalently linked to a ribose or deoxyribose sugar. Exemplary nucleosides include adenosine, guanosine, cytidine, uridine and thymidine. Additional exemplary nucleosides include inosine, 1-methyl inosine, pseudouridine, 5,6-dihydrouridine, ribothymidine, 2N-methylguanosine and N(2),N(2)-dimethylguanosine (also referred to as “rare” nucleosides). The term “nucleotide” refers to a nucleoside having one or more phosphate groups joined in ester linkages to the sugar moiety. Exemplary nucleotides include nucleoside monophosphates, diphosphates and triphosphates. The terms “polynucleotide” and “nucleic acid molecule” are used interchangeably herein and refer to a polymer of nucleotides joined together by a phosphodiester or phosphorothioate linkage between 5′ and 3′ carbon atoms.

The term “RNA” or “RNA molecule” or “ribonucleic acid molecule” refers to a polymer of ribonucleotides (e.g., 2, 3, 4, 5, 10, 15, 20, 25, 30, or more ribonucleotides). An RNA nucleotide refers to a single ribonucleotide. The term “DNA” or “DNA molecule” or “deoxyribonucleic acid molecule” refers to a polymer of deoxyribonucleotides. A DNA nucleotide refers to a single deoxyribonucleotide. As used herein, the term “DNA-like” refers to a conformation of, e.g. a modified nucleoside or nucleotide which is similar to the conformation of a corresponding unmodified DNA unit. For example, a DNA-like nucleotide can refer to a conformation of a modified deoxyribonucleotide similar to a corresponding unmodified deoxyribonucleotide. Examples of DNA-like nucleotides include, without limitation, e.g., 2′-deoxyribonucleotides, 2′-deoxy-2′-substituted arabinonucleotides (e.g., 2′-deoxy-2′-fluoroarabinonucleotides, also known in the art as 2′F-ANA or FANA), and corresponding phosphorothioate analogs. As used herein, the term “RNA-like” refers to a conformation of, e.g. a modified nucleoside or nucleotide which is similar to the conformation of a corresponding unmodified RNA unit. RNA-like conformations can adopt an A-form helix while DNA-like conformations adopt a B-form helix. Examples of RNA-like nucleotides include, without limitation, e.g., 2′-substituted-RNA nucleotides (e.g., 2′-fluoro-RNA nucleotides also known in the art as 2′F-RNA), locked nucleic acid (LNA) nucleotides (also known in the art as bridged nucleic acids or bicyclic nucleotides), 2′-fluoro-4′-thioarabinonucleotide (also known in the art as 4'S-FANA nucleotides), 2′-O-alkyl-RNA, and corresponding phosphorothioate analogs.

DNA and RNA can be synthesized naturally (e.g., by DNA replication or transcription of DNA, respectively). RNA can be post-transcriptionally modified. DNA and RNA can also be chemically synthesized. DNA and RNA can be single-stranded (i.e., ssRNA and ssDNA, respectively) or multi-stranded (e.g., double stranded, i.e., dsRNA and dsDNA, respectively). “mRNA” or “messenger RNA” is single-stranded RNA that specifies the amino acid sequence of one or more polypeptide chains. This information is translated during protein synthesis when ribosomes bind to the mRNA.

As used herein, the term “small interfering RNA” (“siRNA”)(also referred to in the art as “short interfering RNAs”) refers to an RNA (or RNA analog) comprising between about 10-50 nucleotides (or nucleotide analogs) which is capable of directing or mediating RNA interference. In one embodiment, a siRNA comprises between about 15-30 nucleotides or nucleotide analogs, or between about 16-25 nucleotides (or nucleotide analogs), or between about 18-23 nucleotides (or nucleotide analogs), or between about 19-22 nucleotides (or nucleotide analogs)(e.g., 19, 20, 21 or 22 nucleotides or nucleotide analogs). The term “short” siRNA refers to a siRNA comprising about 21 nucleotides (or nucleotide analogs), for example, 19, 20, 21 or 22 nucleotides. The term “long” siRNA refers to a siRNA comprising about 24-25 nucleotides, for example, 23, 24, 25 or 26 nucleotides. Short siRNAs can, in some instances, include fewer than 19 nucleotides, e.g., 16, 17 or 18 nucleotides, provided that the shorter siRNA retains the ability to mediate RNAi. Likewise, long siRNAs can, in some instances, include more than 26 nucleotides, provided that the longer siRNA retains the ability to mediate RNAi absent further processing, e.g., enzymatic processing, to a short siRNA.

The term “nucleotide analog” or “altered nucleotide” or “modified nucleotide” refers to a non-standard nucleotide, including non-naturally occurring ribonucleotides or deoxyribonucleotides. Exemplary modified nucleotides are modified at any position so as to alter certain chemical properties of the nucleotide yet retain the ability of the modified nucleotide to perform its intended function. Examples of positions of the nucleotide which can be derivatized include the 5 position, e.g., 5-(2-amino)propyl uridine, 5-bromo uridine, 5-propyne uridine, 5-propenyl uridine, etc.; the 6 position, e.g., 6-(2-amino)propyl uridine; the 8-position for adenosine and/or guanosines, e.g., 8-bromo guanosine, 8-chloro guanosine, 8-fluoroguanosine, etc. Modified nucleotides also include deaza nucleotides, e.g., 7-deaza-adenosine; O- and N-modified (e.g., alkylated, e.g., N6-methyl adenosine, or as otherwise known in the art) nucleotides; and other heterocyclically modified nucleotides such as those described in Herdewijn, Antisense Nucleic Acid Drug Dev., 2000 Aug. 10(4):297-310.

Modified nucleotides can also comprise modifications to the sugar portion of the nucleotides. For example, the 2′ OH-group may be replaced by a group selected from H, OR, R, F, Cl, Br, I, SH, SR, NH₂, NHR, NR₂, COOR, or OR, wherein R is substituted or unsubstituted C₁-C₆ alkyl, alkenyl, alkynyl, aryl, etc. For another example, the ribose sugar can be replaced with a bicyclic or tricylic moiety, such as in Locked Nucleic Acid, constrained ethyl, tricyclo-DNA (tcDNA), or other bridged or bicyclic modifications. Other possible modifications include those described in U.S. Pat. Nos. 5,858,988, and 6,291,438.

The phosphate group of the nucleotide can also be modified, e.g., by substituting one or more of the oxygens of the phosphate group with sulfur (e.g., phosphorothioates), or by making other substitutions which allow the nucleotide to perform its intended function such as described in Eckstein, Antisense Nucleic Acid Drug Dev. 2000 Apr. 10(2):117-21, Rusckowski et al. Antisense Nucleic Acid Drug Dev. 2000 Oct. 10(5):333-45, Stein, Antisense Nucleic Acid Drug Dev. 2001 Oct. 11(5): 317-25, Vorobjev et al. Antisense Nucleic Acid Drug Dev. 2001 Apr. 11(2):77-85, and U.S. Pat. No. 5,684,143. Certain of the above-referenced modifications (e.g., phosphate group modifications) decrease the rate of hydrolysis of polynucleotides comprising said analogs in vivo or in vitro.

As used herein, the terms “unmodified nucleotide” or “non-modified nucleotide” refers to a nucleotide composed of naturally occurring nucleobases, sugar moieties, and internucleoside linkages. In some embodiments, a non-modified nucleotide is an RNA nucleotide (i.e. β-D-ribonucleoside) or a DNA nucleotide (i.e. β-D-deoxyribonucleoside).

The term “oligonucleotide” refers to a short polymer of nucleotides and/or modified nucleotides. As discussed above, the oligonucleotides can be linked with non-phosphodiester linkages, which result in a lower rate of hydrolysis as compared to an oligonucleotide linked with phosphodiester linkages. For example, the nucleotides of the oligonucleotide can comprise triazole, amide, carbamate, methylenediol, ethylene diol, oxymethylthio, oxyethylthio, oxycarbonyloxy, phosphorodiamidate, phosphoroamidate, phosphonate, and/or phosphorothioate linkages. Alterations or modifications of the oligonucleotide can further include addition of non-nucleotide material to the end(s) of the oligonucleotide or internally (at one or more nucleotides of the oligonucleotide). The oligonucleotide can comprise a lysosome ligand.

As used herein, the term “antisense oligonucleotide” refers to an oligonucleotide molecule, which is capable of binding to RNA inside cells by Watson-Crick base pairing. Depending on the sequence and chemistry of the antisense oligonucleotide, this interaction can lead to silencing of a target gene (i.e. reducing the level of expression of mature mRNA and/or protein from that gene) or activation of a target gene (i.e. increasing the level of expression of mature mRNA and/or protein from that gene). The antisense oligonucleotides of the present disclosure are focused on activating gene expression, which can be done utilizing different mechanisms. Some antisense oligonucleotides are designed to recruit RNase H to cleave their target RNAs. RNase H is a family of non-sequence-specific endonuclease enzymes that catalyze the cleavage of RNA in an RNA/DNA substrate via a hydrolytic mechanism. In certain embodiments, the antisense oligonucleotides of the disclosure trigger RNase H-mediated cleavage of a pre-mRNA target (e.g., EXOC2, Ku80, and Task1 pre-mRNA), which can be compatible with activation of overall target gene expression (e.g., EXOC2, Ku80, and Task1 gene expression). Other antisense oligonucleotides, called steric blockers, are designed not to elicit cleavage of their targets but to block interactions with cellular factors. For example, these cellular factors could modulate splicing, block interactions of noncoding RNAs or of RNA-binding proteins, stabilize mRNA to prolong its half-life, or increase the efficiency of translation of an mRNA.

Antisense oligonucleotides designed to recruit RNase H are often designed as “gapmers.” The term “gapmer” means a chimeric antisense oligonucleotide in which an internal region having a plurality of nucleosides that support RNase H cleavage is positioned between external regions having one or more nucleosides, wherein the nucleosides comprising the internal region are chemically distinct from the nucleoside or nucleosides comprising the external regions. The internal region can be referred to as a “gap segment” and the external regions can be referred to as “wing segments.” “Chimeric antisense oligonucleotide” means an antisense oligonucleotide that has at least two chemically distinct regions.

As used herein, the term “lysosome ligand” is a ligand that interacts with a lysosome. Without being bound to any specific theory, the lysosome ligand interacts with the LAMP-2C protein of the lysosome and triggers the uptake and degradation by the lysosome of a target RNA attached to an oligonucleotide comprising the lysosome ligand. In some embodiments, the lysosome ligand is a poly-G ligand or sequence.

As used herein, a “poly-G ligand” or “poly-G sequence” is a polynucleotide sequence containing two or more G nucleotides and lacks complementarity to the target polynucleotide of the oligonucleotide to which the poly-G sequence is attached.

In some embodiments, the poly-G sequence comprises 2-30 G nucleotides. In some embodiments, the poly-G sequence is 2, 3, 4, 5, 6, 7, 8, 9, 10, 11, 12, 13, 14, or 15 G nucleotides (SEQ ID NO: 57). In some embodiments, the poly-G sequence comprises or consists of 5 G nucleotides. In some embodiments, the poly-G sequence comprises or consists of 6 G nucleotides. In some embodiments, the poly-G sequence comprises or consists of 7 G nucleotides. In some embodiments, the poly-G sequence comprises or consists of 8 G nucleotides. In some embodiments, the poly-G sequence comprises or consists of 9 G nucleotides. In some embodiments, the poly-G sequence comprises or consists of 10 G nucleotides. In some embodiments, the poly-G sequence comprises or consists of 11 G nucleotides. In some embodiments, the poly-G sequence comprises or consists of 12 G nucleotides. In some embodiments, the poly-G sequence comprises or consists of 13 G nucleotides. In some embodiments, the poly-G sequence comprises or consists of 14 G nucleotides. In some embodiments, the poly-G sequence comprises or consists of 15 G nucleotides.

In some embodiments, the poly-G sequence is linked to the 3′ end of an oligonucleotide of the disclosure. In other embodiments, the poly-G sequence is linked to the 5′ end of an oligonucleotide of the disclosure. In other embodiments, the poly-G sequence is linked to the 5′ end and the 3′ end of an oligonucleotide of the disclosure.

In some embodiments, the poly-G sequence is single stranded.

In some embodiments, the poly-G sequence binds to a protein on the surface of a lysosome. In some embodiments, the poly-G sequence binds to a lysosome-associated membrane glycoprotein (LAMP). In some embodiments, the LAMP is LAMP2C.

In some embodiments, the poly-G sequence comprises G nucleotides that are consecutive. In some embodiments, the poly-G sequence comprises or consists of 5-15 consecutive G nucleotides. In some embodiments, the poly-G sequence comprises 1 or more non-G nucleotides (e.g., A, T, U, or C) within the poly-G sequence. In some embodiments, the oligonucleotide comprises two or more poly-G sequences with 1 or more non-G nucleotides (e.g., A, T, U, or C) between the two or more poly-G sequences. For example, but in no way limiting, the oligonucleotide may comprise at the 3′ end (from 5′ to 3′), a first poly-G sequence of 5-10 consecutive G nucleotides, one or more non-G nucleotides (e.g., A, T, U, or C), and a second poly-G sequence of 5-10 consecutive G nucleotides.

In some embodiments, the poly-G sequence comprises G nucleotides that are not consecutive.

In some embodiments, the poly-G sequence comprises or consists of (dG)(dG)(dG)(dG)(dG). In some embodiments, the poly-G sequence comprises or consists of (dG)(dG)(dG)(dG)(dG)(dG). In some embodiments, the poly-G sequence comprises or consists of (dG)(dG)(dG)(dG)(dG)(dG)(dG). In some embodiments, the poly-G sequence comprises or consists of (dG)(dG)(dG)(dG)(dG)(dG)(dG)(dG). In some embodiments, the poly-G sequence comprises or consists of (dG)(dG)(dG)(dG)(dG)(dG)(dG)(dG)(dG). In some embodiments, the poly-G sequence comprises or consists of (dG)(dG)(dG)(dG)(dG)(dG)(dG)(dG)(dG)(dG)(SEQ ID NO: 56). In some embodiments, the poly-G sequence comprises or consists of (dG)(dG)(dG)(dN)(dG)(dG)(dG)(dN)(dG), wherein each dN individually corresponds to a deoxyribonucleotide of dA, dT, or dC. In some embodiments, the poly-G sequence comprises or consists of (dG)(dG)(dG)(dN)(dG)(dG)(dG)(dN)(dG)(dG)(SEQ ID NO: 58), wherein each dN individually corresponds to a deoxyribonucleotide of dA, dT, or dC. In some embodiments, the poly-G sequence comprises or consists of (dG)(dG)(dG)(dN)(dG)(dG)(dG)(dN)(dG)(dG) (SEQ ID NO: 58), wherein each dN individually corresponds to a deoxyribonucleotide of dA, dT, or dC.

As used herein, the term “functional moiety” is a moiety that is linked to an oligonucleotide. a functional moiety is linked to the 5′ end or 3′ end of the oligonucleotide. The functional moiety can comprise an N-acetylgalactosamine (GalNAc) moiety and/or a hydrophobic moiety. The hydrophobic moiety can be fatty acids, steroids, secosteroids, lipids, gangliosides, nucleoside analogs, endocannabinoids, vitamins, and a mixture thereof. The steroid can be cholesterol and Lithocholic acid (LCA). The fatty acid can be Eicosapentaenoic acid (EPA), Docosahexaenoic acid (DHA), and Docosanoic acid (DCA).

As used herein, the term “target polynucleotide” is a polynucleotide which has sufficient complementarity to an oligonucleotide of the disclosure to mediate lysosomal targeting via the poly-G sequence. Exemplary target polynucleotides include mRNA and viral RNA.

As used herein, the term “target gene” is a gene whose expression is to be substantially silenced, restored, or increased. In certain embodiments, the target gene expression is silenced, restored, or increased to wild type levels by an oligonucleotide that silenced expression through direct base pairing interactions with the target sequence and via the lysosomal pathway (e.g., an EXOC2, Ku80, and Task1 mRNA target sequence). In certain embodiments, the target gene expression is silenced, restored, or increased to wild type levels through RNA silencing, e.g., by cleaving a transcript corresponding to a target gene or translational repression of the target gene. Without wishing to be bound by theory, cleavage of a target transcript can increase the levels of productive transcription of the target gene. For example, but in no way limiting, an allele of the target gene can be expressed to form pre-mRNA which can be defective (e.g., contain a nucleotide repeat region that contributes to disease). The target gene expression can be restored by cleaving and degrading the defective pre-mRNA derived from the defective allele, thereby freeing transcriptional machinery to trigger transcription of the non-defective target gene allele. The term “non-target gene” is a gene whose expression is not to be substantially increased, restored, or silenced. For example, a target gene of the present disclosure is EXOC2, Ku80, and Task1, and a non-target gene of the present disclosure is a gene that is not EXOC2, Ku80, and Task1. In one embodiment, the polynucleotide sequences of the target and non-target gene (e.g., mRNA encoded by the target and non-target genes) can differ by one or more nucleotides. In another embodiment, the target and non-target genes can differ by one or more polymorphisms (e.g., Single Nucleotide Polymorphisms or SNPs). In another embodiment, the target and non-target genes can share less than 100% sequence identity. In another embodiment, the target and non-target genes can share less than 99%, 98%, 97%, 96%, 95%, 94%, 93%, 92%, 91%, 90%, 89%, 88%, 86%, 85%, 80%, 75%, or 70% sequence identity. In another embodiment, the non-target gene can be a homologue (e.g., an orthologue or paralogue) of the target gene.

The term “antisense activity” means any detectable or measurable activity attributable to the hybridization of an antisense compound to its target nucleic acid. In some embodiments, antisense activity is an increase in the amount or expression of a target nucleic acid or protein encoded by such target nucleic acid. “Antisense compound” means an oligomeric compound that is capable of undergoing hybridization to a target nucleic acid through hydrogen bonding. As used herein, “antisense oligonucleotide” means a single-stranded oligonucleotide having a nucleobase sequence that permits hybridization to a corresponding region or segment of a target nucleic acid.

The term “antisense inhibition” means reduction of target nucleic acid levels in the presence of an antisense oligonucleotide having a sequence that is sufficiently complementary to a target nucleic acid compared to target nucleic acid levels in the absence of the antisense compound. A target nucleic acid can be any nucleic acid. For example, a target nucleic acid of the present disclosure can be a EXOC2, Ku80, Task1, SOD1, or PCSK9 transcript. In certain embodiments, the target nucleic acid is EXOC2, Ku80, Task1, SOD1, or PCSK9 pre-mRNA.

The term “target-recognition sequence” refers to the portion of an antisense compound that recognizes a target nucleic acid. The target-recognition sequence has a nucleobase sequence that permits hybridization to a corresponding region or segment of a target nucleic acid.

The term “conserved region” refers to a portion, or portions, of a nucleic acid sequence that is conserved, i.e. a portion, or portions of the nucleic acid sequence having a similar or identical sequence across species. A conserved region can be computationally identified, e.g., using any sequence alignment software available in the art.

As used herein, the term “sufficiently complementary” means that the antisense compound has a sequence (e.g., an antisense oligonucleotide having a target-recognition sequence), which is sufficient to bind the desired target transcript (e.g., a EXOC2, Ku80, or Task1transcript), and to increase, restore, or silenced expression of the EXOC2, Ku80, or Task1 gene. For example, a target-recognition sequence with at least 90% complementarity to a target nucleic acid sequence (e.g., a portion of a EXOC2, Ku80, or Task1 transcript) can be sufficiently complementary to increase, restore, or silenced expression of the EXOC2, Ku80, or Task1 gene. The term “perfectly complementary” refers to, e.g., a target-recognition sequence with 100% complementarity to a target nucleic acid sequence. Complementary nucleic acid molecules hybridize to each other. The term “hybridization” means the annealing of complementary nucleic acid molecules. In certain embodiments, complementary nucleic acid molecules include an antisense compound and a target nucleic acid.

As used herein, a “region of complementarity” refers to a portion of the antisense oligonucleotide that is complementary to the target transcript (e.g. the EXOC2, Ku80, or Task1 transcript). For example, but in no way limiting, an 18-nucleotide long antisense oligonucleotide can comprise a contiguous 12-nucleotide portion that is complementary to the target transcript. In certain embodiments, the antisense oligonucleotide is complementary to the target transcript over the full length of the antisense oligonucleotide.

As used herein, “administer” or “administration” refers to the act of injecting or otherwise physically delivering a substance as it exists outside the body (e.g., an antisense compound provided herein) into a patient. The antisense oligonucleotides described herein can be administered to the central nervous system of a patient. The central nervous system includes the brain and spinal cord. Administration methods to the central nervous system include, but not limited to, intrathecal, intraventricular or intrastriatal infusion or delivery and/or any other method of physical delivery described herein or known in the art. Intraventricular infusion can comprise administration using an Ommaya reservoir. In some embodiments, the antisense oligonucleotides described herein can be administered to the patient systemically (such as intravenously, subcutaneously, or intramuscularly). These compounds can be designed to cross into the central nervous system, or to be active in other tissues, such as muscle (including skeletal or heart muscle) or pancreas.

When a disease, or a symptom thereof, is being managed or treated, the administration of the substance typically occurs after the onset of the disease or symptoms thereof. When a disease, or symptom thereof, is being prevented, administration of the substance typically occurs before the onset of the disease or symptoms thereof and can be continued chronically to defer or reduce the appearance or magnitude of disease-associated symptoms, e.g., damage to the involved tissues and airways.

As used herein, the term “composition” is intended to encompass a product containing the specified ingredients (e.g., an antisense compound provided herein) in, optionally, the specified amounts, as well as any product which results, directly or indirectly, from combination of the specified ingredients in, optionally, the specified amounts.

“Effective amount” means the amount of active pharmaceutical agent (e.g., an antisense compound of the present disclosure) sufficient to effectuate a desired physiological outcome in an individual in need of the agent. The effective amount can vary among individuals depending on the health and physical condition of the individual to be treated, the taxonomic group of the individuals to be treated, the formulation of the composition, assessment of the individual's medical condition, and other relevant factors.

As used herein, the terms “subject” and “patient” are used interchangeably. As used herein, a subject can be a mammal, such as a non-primate (e.g., cows, pigs, horses, cats, dogs, rats, etc.) or a primate (e.g., monkey and human). In certain embodiments, the term “subject” refers to a vertebrate, such as a mammal. Mammals include, without limitation, humans, non-human primates, wild animals, feral animals, farm animals, sports animals, and pets. In one embodiment, the subject is a mammal, such as a human, having a EXOC2-related disorder, a Ku80-related disorder, and/or a Task1-related disorder. In another embodiment, the subject is a mammal, such as a human, that is at risk for developing a EXOC2-related disorder, aKu80-related disorder, and/or a Task1-related disorder.

As used herein, the term “therapy” refers to any protocol, method and/or agent that can be used in the prevention, management, treatment and/or amelioration of a disease or a symptom related thereto, such as a EXOC2-related disorder, aKu80-related disorder, and/or a Task1-related disorder. In some embodiments, the term “therapy” refers to any protocol, method and/or agent that can be used in the modulation of an immune response to an infection in a subject or a symptom related thereto. In some embodiments, the terms “therapies” and “therapy” refer to a biological therapy, supportive therapy, and/or other therapies useful in the prevention, management, treatment and/or amelioration of a disease or a symptom related thereto, such as a EXOC2-related disorder, a Ku80-related disorder, and/or a Task1-related disorder known to one of skill in the art such as medical personnel. In other embodiments, the terms “therapies” and “therapy” refer to a biological therapy, supportive therapy, and/or other therapies useful in the modulation of an immune response to an infection in a subject or a symptom related thereto known to one of skill in the art.

As used herein, the terms “treat,” “treatment” and “treating” refer to the reduction or amelioration of the progression, severity, and/or duration of a disease or a symptom related thereto, such as a EXOC2-related disorder, a Ku80-related disorder, and/or a Task1-related disorder, resulting from the administration of one or more therapies (including, but not limited to, the administration of one or more prophylactic or therapeutic agents, such as an antisense oligonucleotide provided herein). The term “treating,” as used herein, can also refer to altering the disease course of the subject being treated. Therapeutic effects of treatment include, without limitation, preventing occurrence or recurrence of disease, alleviation of symptom(s), diminishment of direct or indirect pathological consequences of the disease, decreasing the rate of disease progression, amelioration or palliation of the disease state, and remission or improved prognosis.

As used herein, the term “EXOC2” refers to a gene that encodes the protein exocyst complex component 2. The protein EXOC2 is a component of the exocyst complex, a multi-protein complex essential for the polarized targeting of exocytic vesicles to specific docking sites on the plasma membrane. The human EXOC2 gene corresponds to NG_047166.1 of the NCBI RefSeq database.

As used herein, the term “Ku80” refers to a protein encoded by the XRCC5 gene. Ku80 is a component of the Ku heterodimer with Ku70, which binds to DNA double-strand break ends and is required for the non-homologous end joining (NHEJ) pathway of DNA repair. The human XRCC5 gene corresponds to NG_029780.1 of the NCBI RefSeq database. The term Ku80 is used herein to also refer to the gene and mRNA encoding Ku80, Therefore, oligonucleotides that target Ku80, target the Ku80 mRNA (including pre-mRNA).

As used herein, the term “Task1,” or “KCNK3” refers to a gene that encodes the potassium channel subfamily K member 3 protein. Task1 is one of the members of the superfamily of potassium channel proteins containing two pore-forming P domains. The human KCNK3 gene corresponds to NG_033884.1 of the NCBI RefSeq database. Inhibitors of SOD1, such as inclisiran, are used for the treatment of hyperlipidemia or reducing low density lipoprotein cholesterol (LDL-C) in a subject.

As used herein, the term “SOD1” refers to a gene that encodes the superoxide dismutase 1 protein. SOD1 is a mammalian enzyme that catalyzes the removal of superoxide radicals. The human SOD1 gene corresponds NG_008689.1 of the NCBI RefSeq database. Inhibitors of SOD1, such as tofersen, are used for the treatment of amyotrophic lateral sclerosis (ALS) in a subject.

As used herein, the term “PCSK9” refers to a gene that encodes the proprotein convertase subtilisin/kexin type 9 protein. PCSK9 binds to and degrades the receptor for low-density lipoprotein particles (LDL). The human PCSK9 gene corresponds to NG_009061.1 of the NCBI RefSeq database. Inhibitors of PCSK9, such as inclisiran, are used for the treatment of hyperlipidemia or reducing low density lipoprotein cholesterol (LDL-C) in a subject.

### Antisense Compounds

The present disclosure provides an antisense compound that linked to a poly-G sequence as disclosed here, for directing a target polynucleotide to the lysosome of a cell for target degradation.

The present disclosure also provides an antisense compound that is capable of decreasing EXOC2, Ku80, and Task1 gene expression by at least 5%, 10%, 15%, 20%, 25%, 30%, 35%, 40%, 45%, 50%, at least 55%, at least 60%, at least 65%, at least 70%, at least 75%, at least 80%, at least 85%, at least 90%, at least 95%, at least 100%, or more.

In certain embodiments, the antisense compounds that are capable of decreasing a target polynucleotide expression (e.g., EXOC2, Ku80, and Task1 gene expression), have chemically modified subunits arranged in patterns, or motifs, to confer to the antisense compounds properties such as enhanced the activity, increased binding affinity for a target nucleic acid, or resistance to degradation by in vivo nucleases.

In some embodiments, an antisense compound of the present disclosure is an antisense oligonucleotide. Chimeric antisense oligonucleotides typically contain at least one region modified so as to confer increased resistance to nuclease degradation, increased cellular uptake, increased binding affinity for the target nucleic acid, and/or increased activity. A second region of a chimeric antisense compound can optionally serve as a substrate for the cellular endonuclease RNase H, which cleaves the RNA strand of an RNA:DNA duplex. In some embodiments, an antisense compound of the present disclosure is a chimeric antisense oligonucleotide having a gapmer motif In a gapmer, an internal region having a plurality of nucleotides that supports RNase H cleavage is positioned between external regions having a plurality of nucleotides that are chemically distinct from the nucleosides of the internal region.

In some embodiments, the present disclosure provides an antisense oligonucleotide having a target-recognition sequence that is sufficiently complementary to an EXOC2, Ku80, and/or Task1 transcript or portion thereof, to direct cleavage of the EXOC2, Ku80, and Task1 transcript by RNase H. The target-recognition sequence of the antisense oligonucleotide can be the full length of the antisense oligonucleotide, or a portion thereof. In some embodiments, the antisense oligonucleotide comprises a gapmer motif.

In the case of an antisense compound having a gapmer motif, the gap segment generally serves as the substrate for endonuclease cleavage, while the wing segments comprise modified nucleosides. In certain embodiments, the regions of a gapmer are differentiated by the types of sugar moieties comprising each distinct region. The types of sugar moieties that are used to differentiate the regions of a gapmer can in some embodiments include β-D-ribonucleosides, β-D-deoxyribonucleosides, 2′-modified nucleosides (such 2′-modified nucleosides can include 2′-MOE, and 2′-O-CH₃ (i.e., OMe), among others), and bicyclic sugar modified nucleosides (such bicyclic sugar modified nucleosides can include those having a 4′-(CH₂)ₙ-O-2′ bridge, where n=1 or n=2). In some embodiments, the wing segments of the gapmer contain one or more tricyclo-DNA (tcDNA) modifications. In some embodiments, each distinct region comprises uniform sugar moieties. In some embodiments, each wing segment comprises a mixture of different nucleotide modifications. For example, in one embodiment, a LNA modification and a 2′-MOE modification could be used in combination for one antisense compound. In one embodiment, a LNA modification and a 2′-O-Methyl modification could be used in combination for one antisense compound. In one embodiment, a LNA modification and a 2′-deoxy modification could be used in combination for one antisense compound. In one embodiment, a LNA modification and a tricyclo-DNA modification could be used in combination for one antisense compound. In one embodiment, a 2′-MOE modification and a tricyclo-DNA modification could be used in combination for one antisense compound.

The gapmer motif can be described using the formula “A-B-C”, where “A” represents the length of the 5′ wing region, “B” represents the length of the gap region, and “C” represents the length of the 3′ wing region. As such, in some embodiments, an antisense oligonucleotide of the present disclosure has the formula:

A-B-C.

As used herein, a gapmer described as “A-B-C” has a configuration such that the gap segment is positioned immediately adjacent each of the 5′ wing segment and the 3′ wing segment. Thus, no intervening nucleotides exist between the 5′ wing segment and gap segment, or the gap segment and the 3′ wing segment.

In some embodiments, the 5′ wing region represented by “A” comprises from about 0 to about 8 modified nucleotides, e.g., from about 1 to about 6 modified nucleotides. For example, the 5′ wing region represented by “A” can be 0, 1, 2, 3, 4, 5, 6, 7, or 8 nucleotides in length. In some embodiments, the 3′ wing region represented by “C” comprises about 0 to about 8 modified nucleotides, e.g., from about 1 to about 6 modified nucleotides. For example, the 3′ wing region represented by “C” can be 0, 1, 2, 3, 4, 5, 6, 7, or 8 nucleotides in length. In some embodiments, “A” and “C” are the same, in some embodiments, they are different.

In some embodiments, the gap region represented by “B” comprises from about 5 to about 18 DNA nucleotides and/or DNA-like nucleotides, e.g., from about 5 to about 12 DNA nucleotides and/or DNA-like nucleotides. For example, the gap region represented by “B” can be 5, 6, 7, 8, 9, 10, 11, 12, 13, 14, 15, 16, 17, or 18 DNA nucleotides and/or DNA-like nucleotides in length. Thus, an antisense oligonucleotide of the present disclosure having a target-recognition sequence with the formula “A-B-C” include, but are not limited to the following gapmer formats, for example 1-10-1 (i.e., one nucleotide—ten nucleotides—one nucleotide), 1-10-1, 1-11-1, 1-12-1, 2-8-2, 2-9-2, 2-10-2, 2-11-2, 2-12-2, 3-6-3, 3-7-3, 3-8-3, 3-9-3, 3-10-3, 3-11-3, 3-12-3, 4-6-4, 4-7-4, 4-8-4, 4-9-4, 4-10-4, 4-11-4, 4-12-4, 5-6-5, 5-7-5, 5-8-5, 5-9-5, 5-10-5, 5-11-5, 5-12-5, 6-6-6, 6-7-6, 6-8-6, 6-9-6, 6-10-6, 6-11-6, or 6-12-6. The wings can also be of different lengths, such as 1-10-6, 3-9-5, 7-9-2, 4-10-5, or other asymmetric combinations of wing lengths flanking a central DNA gap. In certain embodiments, the gapmer of “A-B-C” is at least 12 nucleotides in length. In certain embodiments, “B” is at least 6 nucleotides in length. A person of skill in the art will be able to identify additional asymmetric combinations of wing lengths.

In certain embodiments, antisense compounds targeted a EXOC2, Ku80, or Task1 nucleic acid possess a 5-9-4 gapmer format. In some embodiments, the antisense compound is an antisense oligonucleotide having a target-recognition sequence with the 5-9-4 format that is sufficiently complementary to a EXOC2, Ku80, or Task1 transcript, or a portion thereof, to direct cleavage of the EXOC2, Ku80, or Task1 transcript by RNase H. In some embodiments, the target-recognition sequence has the formula “A-B-C”, wherein “A” comprises about 2 to 6 modified nucleotides, “B” comprises about 6 to 12 DNA nucleotides and/or DNA-like nucleotides, and “C” comprises about 2 to 6 modified nucleotides. In some embodiments, the target-recognition sequence has the formula “A-B-C”, wherein “A” comprises 5 modified nucleotides, “B” comprises 9 DNA nucleotides and/or DNA-like nucleotides, and “C” comprises 4 modified nucleotides. In some embodiments, the target-recognition sequence has the formula “A-B-C”, wherein “A” comprises 2 to 6 2′-O-(2-methoxyethyl)(MOE) modified nucleotides, “B” comprises 6 to 12 DNA nucleotides and/or DNA-like nucleotides, and “C” comprises 2 to 6 2′-O-(2-methoxyethyl)(MOE) modified nucleotides. In some embodiments, the target-recognition sequence has the formula “A-B-C”, wherein “A” comprises 5 2′-O-(2-methoxyethyl)(MOE) modified nucleotides, “B” comprises 9 DNA nucleotides and/or DNA-like nucleotides, and “C” comprises 4 2′-O-(2-methoxyethyl)(MOE) modified nucleotides.

In some embodiments, antisense compounds that target a EXOC2, Ku80, or Task1 nucleic acid possess a “wingmer” motif. The wingmer motif can be described using the formula “X-Y” or “Y-X”, where “X” represents the length of the wing region, and “Y” represents the length of the gap region. As such, in some embodiments, an antisense oligonucleotide of the present disclosure has the formula:

X-Y, or

Y-X.

As used herein, a wingmer described as “X-Y” or “Y-X” has a configuration such that the gap segment is positioned immediately adjacent to the wing segment. Thus, no intervening nucleotides exist between the wing segment and the gap segment. Non-limiting examples of wingmer configurations of an antisense compound of the present disclosure include, e.g., 1-15, 1-17, 1-19, 2-15, 2-17, 2-19, 2-22, 3-13, 3-17, 3-20, 3-21, 3-22, 4-12, 4-14, 4-16, 4-18, 4-19, 4-21, 5-11, 5-13, 5-14, 5-15, 5-16, 5-18, or 5-20.

In some embodiments, antisense compounds targeted to a EXOC2, KU80, or TASK1 nucleic acid possess a gap-widened motif. As used herein, “gap-widened” refers to an antisense compound having a gap segment of 12 or more contiguous DNA nucleotides and/or DNA-like nucleotides adjacent to a wing region. In the case of a gap-widened gapmer, the gapmer comprises a gap region having 12 or more contiguous DNA nucleotides and/or DNA-like nucleotides positioned between and immediately adjacent to the 5′ and 3′ wing segments. In the case of a gap-widened wingmer, the wingmer comprises a gap region having 12 or more contiguous DNA nucleotides and/or DNA-like nucleotides positioned immediately adjacent to the wing segment.

A nucleoside is a base-sugar combination. The nucleobase (also known as base) portion of the nucleoside is normally a heterocyclic base moiety. Nucleotides are nucleosides that further include a phosphate group covalently linked to the sugar portion of the nucleoside. For those nucleosides that include a pentofuranosyl sugar, the phosphate group can be linked to the 2′, 3′ or 5′ hydroxyl moiety of the sugar. Oligonucleotides are formed through the covalent linkage of adjacent nucleosides to one another, to form a linear polymeric oligonucleotide. Within the oligonucleotide structure, the phosphate groups are commonly referred to as forming the internucleoside linkages of the oligonucleotide.

Modifications to antisense compounds encompass substitutions or changes to internucleoside linkages, sugar moieties, or nucleobases. Modified antisense compounds are often preferred over native forms because of desirable properties such as, for example, enhanced cellular uptake, enhanced affinity for nucleic acid target, increased stability in the presence of nucleases, or increased inhibitory activity.

Chemically modified nucleosides can also be employed to increase the binding affinity of a shortened or truncated antisense oligonucleotide for its target nucleic acid. Consequently, comparable results can often be obtained with shorter antisense compounds that have such chemically modified nucleosides.

The naturally occurring internucleoside linkage of RNA and DNA is a 3′ to 5′ phosphodiester linkage. Antisense compounds having one or more modified, i.e. non-naturally occurring, internucleoside linkages are often selected over antisense compounds having naturally occurring internucleoside linkages because of desirable properties such as, for example, enhanced cellular uptake, enhanced affinity for target nucleic acids, and increased stability in the presence of nucleases.

Oligonucleotides having modified internucleoside linkages include internucleoside linkages that retain a phosphorus atom as well as internucleoside linkages that do not have a phosphorus atom. Representative phosphorus containing internucleoside linkages include, but are not limited to, phosphodiesters, phosphotriesters, methylphosphonates, phosphoramidates, and phosphorothioates. Methods of preparation of phosphorous-containing and non-phosphorous-containing linkages are well known.

In certain embodiments, antisense compounds targeted to a EXOC2, KU80, or TASK1 nucleic acid comprise one or more modified internucleoside linkages. In certain embodiments, the modified internucleoside linkages are phosphorothioate linkages. In certain embodiments, each internucleoside linkage of an antisense compound is a phosphorothioate internucleoside linkage.

Antisense compounds of the disclosure can optionally contain one or more nucleosides wherein the sugar group has been modified. Such sugar-modified nucleosides can impart enhanced nuclease stability, increased binding affinity or some other beneficial biological property to the antisense compounds. In certain embodiments, nucleosides comprise a chemically modified ribofuranose ring moieties. Examples of chemically modified ribofuranose rings include without limitation, addition of substituent groups (including 5′ and 2′ substituent groups, bridging of ring atoms to form bicyclic nucleic acids (BNA), replacement of the ribosyl ring oxygen atom with S, N(R), or C(R¹)(R²)(R═H, C₁-C₁₂ alkyl or a protecting group) and combinations thereof. Examples of chemically modified sugars include 2′-F-5′-methyl substituted nucleoside (see PCT International Application WO 2008/101157 Published on Aug. 21, 2008 for other disclosed 5′,2′-bis substituted nucleosides) or replacement of the ribosyl ring oxygen atom with S with further substitution at the 2′-position (see published U.S. Patent Application US2005-0130923, published on Jun. 16, 2005) or alternatively 5′-substitution of a BNA (see PCT International Application WO 2007/134181 Published on Nov. 22, 2007 wherein LNA is substituted with for example a 5′-methyl or a 5′-vinyl group).

Examples of nucleosides having modified sugar moieties include without limitation nucleosides comprising 5′-vinyl, 5′-methyl(R or S), 4′-S, 2′-F (i.e., 2′-fluoro), 2′-OCH₃ (i.e., 2′-O-methyl) and 2′-O(CH₂)₂OCH₃ (i.e., 2′-O-methoxyethyl) substituent groups. The substituent at the 2′ position can also be selected from allyl, amino, azido, thio, 0-allyl, 0-C1-C10 alkyl, OCF₃, O(CH₂)₂SCH₃, O(CH₂)₂-O-N(Rₘ)(Rₙ), and O-CH₂-C(═O)—N(Rₘ)(Rₙ), where each Rₘ and Rₙ is, independently, H or substituted or unsubstituted C₁-C₁₀ alkyl. 2′-modified nucleotides are useful in the present disclosure, for example, 2′-O-methyl RNA, 2′-0-methoxyethyl RNA, 2′-fluoro RNA, and others envisioned by one of ordinary skill in the art.

Examples of bicyclic nucleic acids (BNAs) include without limitation nucleosides comprising a bridge between the 4′ and the 2′ ribosyl ring atoms. A BNA comprising a bridge between the 4′ and 2′ ribosyl ring atoms can be referred to as a locked nucleic acid (LNA), and is often referred to as inaccessible RNA. As used herein, the term “locked nucleotide” or “locked nucleic acid (LNA)” comprises nucleotides in which the 2′ deoxy ribose sugar moiety is modified by introduction of a structure containing a heteroatom bridging from the 2′ to the 4′ carbon atoms. The term “non-locked nucleotide” comprises nucleotides that do not contain a bridging structure in the ribose sugar moiety. Thus, the term comprises DNA and RNA nucleotide monomers (phosphorylated adenosine, guanosine, uridine, cytidine, deoxyadenosine, deoxyguanosine, deoxythymidine, deoxycytidine) and derivatives thereof as well as other nucleotides having a 2′-deoxy-erythro-pentofuranosyl sugar moiety or a ribo-pentofuranosyl moiety. In certain embodiments, antisense compounds provided herein include one or more BNA nucleosides wherein the bridge comprises one of the formulas: 4′-(CH₂)-O-2′ (LNA); 4′-(CH₂)-S-2′; 4′-(CH₂)-O-2′ (LNA); 4′-(CH₂)2-O-2′ (ENA); 4′-C(CH₃)2-O-2′ (see PCT/US2008/068922); 4′-CH(CH₃)-O-2′ and 4′-CH(CH₂OCH₃) -O-2′ (see U.S. Pat. No. 7,399,845, issued on Jul. 15, 2008); 4′-CH₂-N(OCH₃)-2′ (see PCT/US2008/064591); 4′-CH₂-O-N(CH₃)-2′ (see published U.S. Patent Application US2004-0171570, published Sep. 2, 2004); 4′-CH₂-N(R)-O-2′ (see U.S. Pat. No. 7,427,672, issued on Sep. 23, 2008); 4′-CH₂-C(CH₃)-2′ and 4′-CH₂-C(═CH₂)-2′ (see PCT/US2008/066154); and wherein R is, independently, H, C1-C12 alkyl, or a protecting group. Each of the foregoing BNAs include various stereochemical sugar configurations including for example α-L-ribofuranose and β-D-ribofuranose (see PCT international application PCT/DK98/00393, published on Mar. 25, 1999 as WO 99/14226).

In some embodiments, antisense compounds provided herein include one or more 2′, 4′-constrained nucleotides. For example, antisense compounds provided by the present disclosure include those having one or more constrained ethyl (cEt) or constrained methoxyethyl(cMOE) nucleotides. In some embodiments, antisense compounds provided herein are antisense oligonucleotides comprising one or more constrained ethyl (cEt) nucleotides. The terms “constrained ethyl” and “ethyl-constrained” are used interchangeably.

In certain embodiments, nucleosides are modified by replacement of the ribosyl ring with a sugar surrogate. Such modification includes without limitation, replacement of the ribosyl ring with a surrogate ring system (sometimes referred to as DNA analogs) such as a morpholino ring, a cyclohexenyl ring, a cyclohexyl ring or a tetrahydropyranyl ring such as one having one of the formula:

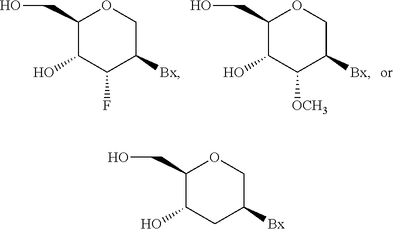

In certain embodiments, antisense oligonucleotides may comprise morpholino rings joined by phosphorodiamidate linkages. These may be referred to as PMO oligomers or phosphorodiamidate morpholino oligomers. In certain such embodiments, the backbone of these oligonucleotides may be uncharged. In other embodiments, one or more of the phosphorodiamidate linkages may comprise a charged moiety.

Many other bicyclo and tricyclo sugar surrogate ring systems are also known in the art that can be used to modify nucleosides for incorporation into antisense compounds (see for example review article: Leumann, J. C, Bioorganic &Medicinal Chemistry, 2002, 10, 841-854; Ito, K. R.; Obika, S., Recent Advances in Medicinal Chemistry of Antisense Oligonucleotides. In Comprehensive Medicinal Chemistry, 3rd edition, Elsevier: 2017). Such ring systems can undergo various additional substitutions to enhance activity.

Methods for the preparations of modified sugars are well known to those skilled in the art. In nucleotides having modified sugar moieties, the nucleobase moieties (natural, modified or a combination thereof) are maintained for hybridization with an appropriate nucleic acid target.

In certain embodiments, antisense compounds targeted to a EXOC2, Ku80, or Task1 nucleic acid comprise one or more kinds of modified nucleotides. In one embodiment, antisense compounds targeted to a EXOC2, Ku80, or Task1 nucleic acid comprise 2′-modified nucleotides. In one embodiment, antisense compounds targeted to a EXOC2, Ku80, or Task1 nucleic acid comprise a 2′-O-methyl RNA, a 2′-O-methoxyethyl RNA, or a 2′-fluoro RNA. In one embodiment, antisense compounds targeted to a EXOC2, Ku80, or Task1 nucleic acid comprise tricyclo-DNA (tcDNA). Tricyclo-DNA belongs to a class of constrained DNA analogs that display improved hybridizing capacities to complementary RNA, see, e.g., Ittig et al., Nucleic Acids Res. 32:346-353 (2004); Ittig et al., Prague, Academy of Sciences of the Czech Republic. 7:21-26 (Coll. Symp. Series, Hocec, M., 2005); Ivanova et al., Oligonucleotides 17:54-65 (2007); Renneberg et al., Nucleic Acids Res. 30:2751-2757 (2002); Renneberg et al., Chembiochem. 5:1114-1118 (2004); and Renneberg et al., JACS. 124:5993-6002 (2002). In one embodiment, antisense compounds targeted to a EXOC2, Ku80, or Task1 nucleic acid comprise a locked nucleotide, an ethyl-constrained nucleotide, or an alpha-L-locked nucleic acid. Various alpha-L-locked nucleic acids are known by those of ordinary skill in the art, and are described in, e.g., Sorensen et al., J Am. Chem. Soc. (2002) 124(10):2164-2176.

In certain embodiments, the antisense compounds targeting a EXOC2, Ku80, or Task1 nucleic acid are fully chemically modified, i.e., every nucleotide is chemically modified. In certain embodiments, every nucleotide comprises a 2′-O-(2-methoxyethyl)(MOE) modification. In certain embodiments, every nucleotide comprises a tricyclo-DNA modification. In certain embodiments, the antisense compounds targeting a EXOC2, Ku80, or Task1 nucleic acid comprise a mixture of tricyclo-DNA modifications and 2′-O-(2-methoxyethyl)(MOE) modifications, wherein every nucleotide of the antisense compounds is either tcDNA or MOE.

In certain embodiments, antisense compounds targeted to a EXOC2, Ku80, or Task1 nucleic acid comprise one or more modified nucleotides having modified sugar moieties. In some embodiments, the modified nucleotide is a locked nucleotide. In certain embodiments, the locked nucleotides are arranged in a gapmer motif, e.g. a 3-9-3 gapmer format wherein 9 non-locked nucleotides are flanked by 3 locked nucleotides on each side.

Nucleobase (or base) modifications or substitutions are structurally distinguishable from, yet functionally interchangeable with, naturally occurring or synthetic unmodified nucleobases. Both natural and modified nucleobases are capable of participating in hydrogen bonding. Such nucleobase modifications can impart nuclease stability, binding affinity or some other beneficial biological property to antisense compounds. Modified nucleobases include synthetic and natural nucleobases such as, for example, 5-methylcytosine (5-me-C). Certain nucleobase substitutions, including 5-methylcytosine substitutions, are useful for increasing the binding affinity of an antisense compound for a target nucleic acid. For example, 5-methylcytosine substitutions have been shown to increase nucleic acid duplex stability by 0.6-1.2° C. (Sanghvi, Y. S., Crooke, S. T. and Lebleu, B., eds., Antisense Research and Applications, CRC Press, Boca Raton, 1993, pp. 276-278).

Additional modified nucleobases include 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl (—C≡C—CH₃) uracil and cytosine and other alkynyl derivatives of pyrimidine bases, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo such as 5-bromo, 5-trifluoromethyl and other 5-substituted uracils and cytosines, 7-methylguanine and 7-methyladenine, 2-F-adenine, 2-amino-adenine, 8-azaguanine and 8-azaadenine, 7-deazaguanine and 7-deazaadenine and 3-deazaguanine and 3-deazaadenine.

Heterocyclic base moieties can also include those in which the purine or pyrimidine base is replaced with other heterocycles, for example 7-deaza-adenine, 7-deazaguanosine, 2-aminopyridine and 2-pyridone. Nucleobases that are useful for increasing the binding affinity of antisense compounds include 5-substituted pyrimidines, 6-azapyrimidines and N-2, N-6 and 0-6 substituted purines, including 2 aminopropyladenine, 5-propynyluracil and 5-propynylcytosine.

In certain embodiments, antisense compounds targeted to a EXOC2, Ku80, or Task1 nucleic acid comprise one or more modified nucleotides having modified sugar moieties. In some embodiments, the modified nucleotide is a locked nucleotide. In certain embodiments, the locked nucleotides are arranged in a gapmer motif, e.g. a 3-9-3 gapmer format wherein 9 non-locked nucleotides are flanked by 3 locked nucleotides on each side. In certain embodiments, antisense compounds targeted to a EXOC2, Ku80, or Task1 nucleic acid comprise one or more modified nucleotides. In some embodiments, the modified nucleotide is 5-methylcytosine. In certain embodiments, each cytosine is a 5-methylcytosine. In some embodiments, the modified nucleotide is a 2′-O-(2-methoxyethyl)(MOE) modified nucleotide. In certain embodiments, the 2′-O-(MOE) modified nucleotides are arranged in a gapmer motif, e.g. a 5-9-4 gapmer format wherein 9 non-2′-O-(MOE) modified nucleotides are flanked by 4 or 5 2′-O-(MOE) modified nucleotides on one or both sides. In certain embodiments, antisense compounds targeted to a EXOC2, Ku80, or Task1 nucleic acid comprise a steric blocking chemical modification format. In some embodiments of the steric blocking chemical modification format, every nucleotide of the antisense compound is a 2′-O-(2-methoxyethyl) (MOE) modified nucleotide. In some embodiments of the steric blocking chemical modification format, every nucleotide of the antisense compound is a tricyclo-DNA modified nucleotide. In some embodiments of the steric blocking chemical modification format, the antisense compound comprises at least one MOE modified nucleotide and at least one tricyclo-DNA modified nucleotide. Many different chemical modification patterns steric blocking antisense oligonucleotides are envisioned. For example, but in no way limiting, the steric blocking antisense oligonucleotide can comprise a mixture of different types of modifications, such as a mixture of 2′-O-(2-methoxyethyl) modifications, LNA modifications, tricyclo-DNA modifications, and DNA modifications where the DNA stretches are four nucleotides or less.

In some embodiments, an antisense compound of the present disclosure directs cleavage of an EXOC2, Ku80, or Task1 transcript by RNase H. In such embodiments, the antisense compound can be referred to as an RNase H-dependent antisense compound. In some embodiments the antisense compound is an RNase H-dependent antisense oligonucleotide. In some embodiments, an antisense oligonucleotide of the present disclosure is an RNase H-dependent antisense oligonucleotide, and can be a single-stranded, chemically modified oligonucleotide that binds to a complementary sequence in the target transcript (e.g., a EXOC2, Ku80, or Task1 transcript). An RNase H-dependent antisense oligonucleotide of the present disclosure reduces expression of a target gene by RNase H-mediated cleavage of the target transcript, and by inhibition of translation by steric blockade of ribosomes. In some embodiments, an antisense compound of the present disclosure is capable of mediating cleavage of at least 50%, at least 55%, at least 60%, at least 65%, at least 70%, at least 75%, at least 80%, at least 85%, at least 90%, at least 95% or more of EXOC2, Ku80, or Task1 transcripts by RNase-H. In one embodiment, the antisense compound is capable of mediating cleavage of at least 80% of EXOC2, Ku80, or Task1 transcripts by RNase-H. In one embodiment, the antisense compound is capable of mediating cleavage of at least 90% of EXOC2, Ku80, or Task1 transcripts by RNase-H.

In certain embodiments, an antisense compound that targets a EXOC2, Ku80, or Task1 transcript is from about 6 to about 24 subunits in length. In other embodiments, the antisense compound that targets an EXOC2, Ku80, or Task1 transcript is from about 8 to about 80 subunits in length. For example, the antisense compounds are 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, or 80 linked subunits in length, or a range defined by any two of the above values. In some embodiments, the antisense compounds are less than 40 linked subunits in length. In some embodiments, the antisense compounds are from about 10 to about 30 linked subunits in length. In some embodiments, the antisense compounds are from about 12 to about 25 linked subunits in length. In some embodiments, the antisense compounds are from about 15 to about 20 linked subunits in length. In some embodiments, the antisense compound is an antisense oligonucleotide that targets a EXOC2, Ku80, or Task1 transcript, and the linked subunits are linked nucleotides.

In certain embodiments antisense compounds targeted to an EXOC2, Ku80, or Task1 transcript can be shortened or truncated. For example, a single subunit can be deleted from the 5′ end (5′ truncation), or alternatively from the 3′ end (3′ truncation). A shortened or truncated antisense compound targeted to a EXOC2, Ku80, or Task1 transcript can have two subunits deleted from the 5′ end, or alternatively can have two subunits deleted from the 3′ end, of the antisense compound. Alternatively, the deleted nucleosides can be dispersed throughout the antisense compound, for example, in an antisense compound having one nucleoside deleted from the 5′ end and one nucleoside deleted from the 3′ end.

When a single additional subunit is present in a lengthened antisense compound, the additional subunit can be located at the 5′ or 3′ end of the antisense compound. When two or more additional subunits are present, the added subunits can be adjacent to each other, for example, in an antisense compound having two subunits added to the 5′ end (5′ addition), or alternatively to the 3′ end (3′ addition), of the antisense compound. Alternatively, the added subunits can be dispersed throughout the antisense compound, for example, in an antisense compound having one subunit added to the 5′ end and one subunit added to the 3′ end.

It is possible to increase or decrease the length of an antisense compound, such as an antisense oligonucleotide, and/or introduce mismatch bases without eliminating activity. For example, in Woolf et al. (Proc. Natl. Acad. Sci. USA 89:7305-7309, 1992), a series of antisense oligonucleotides 13-25 nucleobases in length were tested for their ability to induce cleavage of a target RNA in an oocyte injection model. Antisense oligonucleotides 25 nucleobases in length with 8 or 11 mismatch bases near the ends of the antisense oligonucleotides were able to direct specific cleavage of the target mRNA, albeit to a lesser extent than the antisense oligonucleotides that contained no mismatches. Similarly, target specific cleavage was achieved using 13 nucleobase antisense oligonucleotides, including those with 1 or 3 mismatches.

In certain embodiments, the antisense oligonucleotide comprises the formula:

A-B-C, wherein:
A comprises from about 0 to about 18 modified nucleotides;
B comprises from about 0 to about 4 deoxyribonucleic acid (DNA) nucleotides and/or DNA-like nucleotides; and
C comprises from about 0 to about 18 modified nucleotides;
and the overall length of the antisense oligonucleotide is about 10 to about 30 nucleotides.
   Antisense oligonucleotides that contain 4 or fewer DNA and/or DNA-like nucleotides in “B” should not recruit RNase H and direct cleavage of a target. In these instances, the antisense oligonucleotide is not a gapmer format, but can rather act as a steric blocker.

### Branched Antisense Compounds

The present disclosure also provides branched antisense compounds comprising two or more target-recognition sequences that targets a portion of a EXOC2, Ku80, or Task1 nucleic acid. A branched antisense compound of the present disclosure can be, e.g., a branched antisense oligonucleotide compound.

As used herein, the term “branched antisense compound” or “branched antisense oligonucleotide” refers to two or more antisense compounds or antisense oligonucleotides that are connected together.

In one embodiment, a branched oligonucleotide compound comprises two or more target-recognition sequences, wherein the target-recognition sequences are connected to one another by one or more moieties selected from a linker, a spacer, and a branching point. Target-recognition sequences are described herein. In some embodiments, the branched oligonucleotide compound comprises 2, 3, 4, 5, 6, 7, 8, or more target-recognition sequences, wherein each target-recognition sequences comprises a 5′ end and a 3′ end, and each target-recognition sequence is independently connected to a linker, a spacer, or a branching point at the 5′ end or the 3′ end. In some embodiments, each target-recognition sequence is connected to a linker, a spacer, or a branching point at the 5′ end. In some embodiments, each target-recognition sequence is connected to a linker, a spacer, or a branching point at the 3′ end. In another embodiment, each target-recognition sequence is connected to a linker, a spacer, or a branching point. In some embodiments, each of the target-recognition sequences are antisense compounds and/or oligonucleotides that target a portion of a EXOC2, Ku80, or Task1 nucleic acid.

In some embodiments, a branched oligonucleotide compound of the present disclosure has the formula

L-(N)ₙ

wherein N represents a target-recognition sequence of the present disclosure; n represents an integer, e.g., 2, 3, 4, 5, 6, 7, or 8; and L represents a linker selected from an ethylene glycol chain, an alkyl chain, a peptide, RNA, DNA, a phosphate, a phosphonate, a phosphoramidate, an ester, an amide, a triazole, and any combination thereof.

In some embodiments, a branched oligonucleotide compound of the present disclosure has the formula

L-(N)ₙ

wherein the compound optionally further comprises one or more branching points B, and wherein the compound optionally further comprises one or more spacers S. In such embodiments, each of the one or more branching points B independently represents a polyvalent organic species or derivative thereof, and each of the one or more spacers S is independently selected from an ethylene glycol chain, an alkyl chain, a peptide, RNA, DNA, a phosphate, a phosphonate, a phosphoramidate, an ester, an amide, a triazole, and any combination thereof. In some embodiments, the spacer S is biocleavable. For example, the spacer S could include moieties susceptible to cleavage by nucleases, by proteases, by changes in pH, or by reduction or oxidation. In such embodiments, spacers could include peptides, phosphodiester-linked nucleotides, disulfide bonds, pH-sensitive linkages or other biocleavable moieties. For example, a branched oligonucleotide compound of the present disclosure having the formula L-(N)n has a structure, not to be limited in any fashion, e.g.,

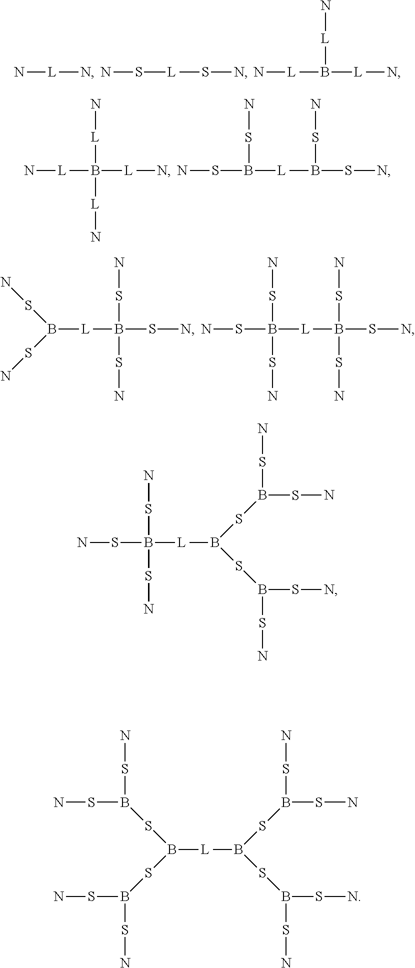

### Target-Recognition Sequences

The present disclosure provides an antisense oligonucleotide comprising a target-recognition sequence that targets a portion of a EXOC2, Ku80, or Task1 nucleic acid. In certain embodiments, an antisense oligonucleotide has a nucleobase sequence that, when written in the 5′ to 3′ direction, comprises the reverse complement of a portion of a EXOC2, Ku80, or Task1 nucleic acid. In certain such embodiments, an antisense oligonucleotide has a nucleobase sequence that, when written in the 5′ to 3′ direction, comprises the reverse complement of a portion of a EXOC2, Ku80, or Task1 nucleic acid.

In certain embodiments, a target region is a structurally defined region of a EXOC2, Ku80, or Task1 nucleic acid. For example, a target region can encompass a 3′ untranslated region (UTR), a 5′ untranslated region (UTR), an exon, an intron, an exon/intron junction, a coding region, a translation initiation region, translation termination region, or other defined nucleic acid region, for example, an open reading frame, or the junction between an open reading frame and an untranslated region and any combinations thereof. The structurally defined regions for EXOC2, Ku80, and Task1 can be obtained by accession number from sequence databases such as NCBI and such information is incorporated herein by reference. In certain embodiments, a target region can encompass the sequence from a 5′ target site of one target segment within the target region to a 3′ target site of another target segment within the same target region.

Targeting includes determination of at least one target segment to which an antisense oligonucleotide hybridizes, such that a desired effect occurs. In certain embodiments, the desired effect is an increase in transcript target nucleic acid levels, i.e., an increase in EXOC2, Ku80, or Task1 transcript levels. In certain embodiments, the desired effect is an increase of levels of protein encoded by the target nucleic acid or a phenotypic change associated with the target nucleic acid, e.g., an increase in the level of EXOC2, Ku80, or Task1 protein.

A target region can contain one or more target segments. Multiple target segments within a target region can be overlapping. Alternatively, they can be non-overlapping. In certain embodiments, target segments within a target region are separated by no more than about 300 nucleotides. In certain embodiments, target segments within a target region are separated by a number of nucleotides that is about 250, 200, 150, 100, 90, 80, 70, 60, 50, 40, 30, 20, or 10 nucleotides on the target nucleic acid, or is a range defined by any two of the preceding values. In certain embodiments, target segments within a target region are separated by no more than about 5 nucleotides on the target nucleic acid. In certain embodiments, target segments are contiguous.

Suitable target segments can be found within a 5′ UTR, a coding region, a 3′ UTR, an intron, an exon, and/or an exon/intron junction. Target segments containing a start codon or a stop codon are also suitable target segments. A suitable target segment can specifically exclude a certain structurally defined region such as the start codon or stop codon. In certain embodiments, the target segments are found in intron 1 of the EXOC2, Ku80, and Task1 gene. In certain embodiments, the target segments are found upstream (5′) of a nucleotide repeat region in intron 1 of the EXOC2, Ku80, and Task1 gene. In certain embodiments, the target segments are found downstream (3′) of a nucleotide repeat region in intron 1 of the EXOC2, Ku80, and Task1 gene. In certain embodiments, the target segments are found upstream or downstream of a nucleotide repeat region in intron 1 of the EXOC2, Ku80, and Task1 gene and are unique in a human genome (i.e., the target segment nucleic acid sequence is found only once in the human genome).

The determination of suitable target segments can include a comparison of the sequence of a target nucleic acid (e.g., EXOC2, Ku80, and Task1) to other sequences throughout the genome. For example, the BLAST algorithm can be used to identify regions of similarity amongst different nucleic acids. This comparison can prevent the selection of antisense oligonucleotide sequences that can hybridize in a non-specific manner to sequences other than a selected target nucleic acid (i.e., non-target or off-target sequences). The determination of suitable target segments can include comparison of the sequences of a target nucleic acid (e.g., a EXOC2, Ku80, and Task1 transcript) across several species. For example, various sequence alignment software can be used to identify regions of similar or identical sequence across species. In certain embodiments, the EXOC2, Ku80, and Task1 transcript target segment nucleic acid sequences are unique in the human genome (i.e., the target segment nucleic acid sequence is found only once in the human genome).

An antisense oligonucleotide and a target nucleic acid (e.g., a EXOC2, Ku80, and Task1transcript or portion thereof) are complementary to each other when a sufficient number of nucleobases of the antisense oligonucleotide can hydrogen bond with the corresponding nucleobases of the target nucleic acid, such that a desired effect will occur (e.g., increased expression of a target nucleic acid, such as a EXOC2, Ku80, and Task1 transcript or portion thereof).

Non-complementary nucleobases between an antisense oligonucleotide and a EXOC2, Ku80, and Task1 nucleic acid can be tolerated provided that the antisense oligonucleotide remains able to specifically hybridize to a target nucleic acid. Moreover, an antisense oligonucleotide can hybridize over one or more segments of a EXOC2, Ku80, and Task1 nucleic acid such that intervening or adjacent segments are not involved in the hybridization event (e.g., a loop structure, mismatch or hairpin structure).

In certain embodiments, the antisense oligonucleotides provided herein, or a specified portion thereof, are, or are at least, 70%, 80%, 85%, 86%, 87%, 88%, 89%, 90%, 91%, 92%, 93%, 94%, 95%, 96%, 97%, 98%, 99%, or 100% complementary to a EXOC2, Ku80, and Task1 nucleic acid, a target region, target segment, or specified portion thereof.

For example, an antisense oligonucleotide in which 18 of 20 nucleobases of the antisense oligonucleotide are complementary to a target region (e.g., an equal length portion of a EXOC2, Ku80, and Task1 transcript), and would therefore specifically hybridize, would represent 90 percent complementarity. In this example, the remaining noncomplementary nucleobases can be clustered or interspersed with complementary nucleobases and need not be contiguous to each other or to complementary nucleobases. As such, an antisense oligonucleotide which is 18 nucleobases in length having 4 (four) noncomplementary nucleobases which are flanked by two regions of complete complementarity with the target nucleic acid would have 77.8% overall complementarity with the target nucleic acid and would thus fall within the scope of the present disclosure. Percent complementarity of an antisense oligonucleotide with a region of a target nucleic acid can be determined routinely using BLAST programs (basic local alignment search tools) and PowerBLAST programs known in the art (Altschul et al., J. Mol. Biol., 1990, 215, 403 410; Zhang and Madden, Genome Res., 1997, 7, 649 656). Percent homology, sequence identity or complementarity, can be determined by, for example, the Gap program (Wisconsin Sequence Analysis Package, Version 8 for Unix, Genetics Computer Group, University Research Park, Madison Wis.), using default settings, which uses the algorithm of Smith and Waterman (Adv. Appl. Math., 1981, 2, 482 489).

In certain embodiments, the antisense oligonucleotides provided herein, or specified portions thereof, are fully complementary (i.e. 100% complementary) to a target nucleic acid, or specified portion thereof. For example, antisense oligonucleotide can be fully complementary to a EXOC2, Ku80, or Task1 nucleic acid, or a target region, or a target segment or target sequence thereof. As used herein, “fully complementary” or “perfectly complementary” means each nucleobase of an antisense oligonucleotide is capable of precise base pairing with the corresponding nucleobases of a target nucleic acid. For example, a 20 nucleobase antisense oligonucleotide is perfectly complementary to a target sequence that is 400 nucleobases long, so long as there is a corresponding 20 nucleobase portion of the target nucleic acid that is fully complementary to the antisense oligonucleotide. Perfectly complementary can also be used in reference to a specified portion of the first and/or the second nucleic acid. For example, a 20 nucleobase portion of a 30 nucleobase antisense oligonucleotide can be “fully complementary” to a target sequence that is 400 nucleobases long. The 20 nucleobase portion of the 30 nucleobase oligonucleotide is fully complementary to the target sequence if the target sequence has a corresponding 20 nucleobase portion wherein each nucleobase is complementary to the 20 nucleobase portion of the antisense oligonucleotide. At the same time, the entire 30 nucleobase antisense oligonucleotide may or may not be fully complementary to the target sequence, depending on whether the remaining 10 nucleobases of the antisense oligonucleotide are also complementary to the target sequence.

In one aspect, the disclosure provides an antisense oligonucleotide comprising a region of complementarity to an intron of a EXOC2, Ku80, and Task1 transcript, wherein the antisense oligonucleotide does not comprise a region of complementarity to another site in a human genome (i.e., the target sequence of the antisense oligonucleotide is unique in a human genome).

In another aspect, the disclosure provides an antisense oligonucleotide comprising a region of complementarity to intron 1 of a EXOC2, Ku80, and Task1 transcript, wherein the antisense oligonucleotide does not comprise a region of complementarity to another site in a human genome (i.e., the target sequence of the antisense oligonucleotide is unique in a human genome).

In certain embodiments, the antisense oligonucleotide comprises a sequence modification pattern of
[Xs]ₐ[Xs]_{b}[sX]_{c}, wherein
“a” represents an integer between 0-8;
“b” represents an integer between 6-18;
“c” represents an integer between 0-8;
“s” represents a phosphorothioate internucleoside linkage;
“X” is an adenosine, a guanosine, a cytidine, a thymine, or a uracil, wherein X comprises a 2′-O-(2-methoxyethyl) modification or a tricyclo-DNA modification (i.e., each of adenosine, guanosine, cytidine, thymine, and uracil comprises a 2′-O-(2-methoxyethyl) modification or a tricyclo-DNA modification);
“X” is an adenosine, a guanosine, a cytidine, a thymine, or a uracil, wherein X comprises a 2′-deoxy modification (i.e., each of adenosine, guanosine, cytidine, thymine, and uracil comprises a 2′-deoxy modification); and
wherein the sum of a, b, and c is greater than or equal to 12.

In certain embodiments, the antisense oligonucleotide comprises a sequence modification pattern of
XsXsXsXsXsXsXsXsXsXsXsXsXsXsXsXsXsX, wherein
“s” represents a phosphorothioate internucleoside linkage;
“X” is an adenosine, a guanosine, a cytidine, a thymine, or a uracil, wherein X comprises a 2′-O-(2-methoxyethyl) modification or a tricyclo-DNA modification; and
“X” is an adenosine, a guanosine, a cytidine, a thymine, or a uracil, wherein X comprises a 2′-deoxy modification.

In certain embodiments, the antisense oligonucleotide comprises a sequence modification pattern of
XsXsXsXsXsXsXsXsXsXsXsXsXsXsXsXsXsX, wherein
“s” represents a phosphorothioate internucleoside linkage;
“X” is an adenosine, a guanosine, a cytidine, a thymine, or a uracil, wherein X comprises a 2′-O-(2-methoxyethyl) modification; and
“X” is an adenosine, a guanosine, a cytidine, a thymine, or a uracil, wherein X comprises a 2′-deoxy modification.

In certain embodiments, the antisense oligonucleotide comprises a sequence modification pattern of
[Xs]_{d}[sX]ₑ, wherein
“d” represents an integer between 0-40;
“e” represents an integer between 0-40;
“s” represents a phosphorothioate internucleoside linkage;
“X” is an adenosine, a guanosine, a cytidine, a thymine, or a uracil, wherein X comprises a 2′-O-(2-methoxyethyl) modification or a tricyclo-DNA modification; and
wherein the sum of d and e is greater than or equal to 10.

In certain embodiments, the antisense oligonucleotide comprises a sequence modification pattern of
XsXsXsXsXsXsXsXsXsXsXsXsXsXsXsXsXsX, wherein
“s” represents a phosphorothioate internucleoside linkage; and
“X” is an adenosine, a guanosine, a cytidine, a thymine, or a uracil, wherein X comprises a 2′-0-(2-methoxyethyl) modification or a tricyclo-DNA modification.

In certain embodiments, the antisense oligonucleotide comprises a sequence modification pattern of
XsXsXsXsXsXsXsXsXsXsXsXsXsXsXsXsXsX, wherein
“s” represents a phosphorothioate internucleoside linkage; and
“X” is an adenosine, a guanosine, a cytidine, a thymine, or a uracil, wherein X comprises a 2′-O-(2-methoxyethyl) modification.

In certain embodiments, each cytosine is a 5-methylcytosine.

In one aspect, the disclosure provides an antisense oligonucleotide comprising the sequence
GsCsAsAsUsAsCsAsTsGsGsAsTsTsGsGsGsG (SEQ ID NO: 53), wherein
“s” represents a phosphorothioate internucleoside linkage;
“A” is an adenosine comprising a 2′-O-(2-methoxyethyl) modification;
“G” is a guanosine comprising a 2′-O-(2-methoxyethyl) modification;
“C” is a cytidine comprising a 2′-O-(2-methoxyethyl) modification;
“U” is a thymine comprising a 2′-O-(2-methoxyethyl) modification;
“A” is an adenosine comprising a 2′-deoxy modification;
“G” is a guanosine comprising a 2′-deoxy modification;
“C” is a cytidine comprising a 2′-deoxy modification; and
“T” is a thymine comprising a 2′-deoxy modification.

In one aspect, the disclosure provides an antisense oligonucleotide comprising the sequence
GsCsAsAsUsAsCsAsUsGsGsAsUsUsGsGsGsG (SEQ ID NO: 54), wherein
“s” represents a phosphorothioate internucleoside linkage;
“A” is an adenosine comprising a 2′-O-(2-methoxyethyl) modification;
“G” is a guanosine comprising a 2′-O-(2-methoxyethyl) modification;
“C” is a cytidine comprising a 2′-O-(2-methoxyethyl) modification; and
“U” is a thymine comprising a 2′-O-(2-methoxyethyl) modification.

In one aspect, the disclosure provides an antisense oligonucleotide comprising the sequence
GsUsAsCsAsAsAsCsUsCsCsGsGsAsGsAsGsC (SEQ ID NO: 55), wherein
“s” represents a phosphorothioate internucleoside linkage;
“A” is an adenosine comprising a 2′-O-(2-methoxyethyl) modification;
“G” is a guanosine comprising a 2′-O-(2-methoxyethyl) modification;
“C” is a cytidine comprising a 2′-O-(2-methoxyethyl) modification; and
“U” is a thymine comprising a 2′-O-(2-methoxyethyl) modification.

In certain embodiments, the two or more antisense oligonucleotides are linked together through a linker. In certain embodiments, the linker is a cleavable linker. In certain embodiments, the cleavable linker degrades when cleaved. In certain embodiments, the cleavable linker is a nuclease-cleavable linker comprising a phosphodiester linkage. In certain embodiments, the nuclease-cleavable linker comprises from about 2 to about 8 nucleotides in length. In certain embodiments, the nuclease-cleavable linker comprises about 6 nucleotides. In certain embodiments, the cleavable linker is cleaved under reducing conditions or changing pH conditions. In certain embodiments, the cleavable linker is cleaved by an intracellular or endosomal nuclease. In certain embodiments, the cleavable linker is cleaved by an intracellular or endosomal protease.

### Conjugated Antisense Oligonucleotides

Antisense oligonucleotides can be covalently linked to one or more moieties, ligands, sequences, or conjugates which enhance the activity, cellular distribution or cellular uptake of the resulting antisense oligonucleotides. Antisense oligonucleotides can be covalently linked to one or more moieties, ligands, sequences, or conjugates which enhance and/or optimize pharmacokinetic parameters. Various pharmacokinetic parameters are absorbance, concentration of a compound in the body, the degree to which a compound permeates the body, the rate of elimination/clearance of a compound, the volume of plasma cleared of a compound per unit time, and others.

Conjugate groups can include a lysosome ligand or sequence. In some embodiments, the lysosome sequence is a poly-G sequence. In some embodiments, the poly-G sequence is a 2-30 poly-G sequence (SEQ ID NO: 57). In some embodiments, the poly-G sequence is a 2, 3, 4, 5, 6, 7, 8, 9, or 10 poly-G sequence (SEQ ID NO: 57).

Conjugate groups can include hydrophobic moieties. In a certain embodiment, the hydrophobic moiety is selected from the group consisting of fatty acids, steroids, secosteroids, lipids, gangliosides and nucleoside analogs, endocannabinoids, and vitamins. In a certain embodiment, the steroid selected from the group consisting of cholesterol and Lithocholic acid (LCA). In a certain embodiment, the fatty acid selected from the group consisting of Eicosapentaenoic acid (EPA), Docosahexaenoic acid (DHA) and Docosanoic acid (DCA). In a certain embodiment, the vitamin selected from the group consisting of choline, vitamin A, vitamin E, and derivatives or metabolites thereof. In a certain embodiment, the vitamin is selected from the group consisting of retinoic acid and alpha-tocopheryl succinate.

In a certain embodiment, an antisense compound of the disclosure is conjugated to a lipophilic moiety. In one embodiment, the lipophilic moiety is a ligand that includes a cationic group. In certain embodiments, the lipophilic moiety is selected from the group consisting of cholesterol, vitamin E, vitamin K, vitamin A, folic acid, or a cationic dye (e.g., Cy3). In an exemplary embodiment, the lipophilic moiety is a cholesterol. Other lipophilic moieties include cholic acid, adamantane acetic acid, 1-pyrene butyric acid, dihydrotestosterone, 1,3-Bis-O(hexadecyl)glycerol, geranyloxyhexyl group, hexadecylglycerol, borneol, menthol, 1,3-propanediol, heptadecyl group, palmitic acid, myristic acid, 03-(oleoyl)lithocholic acid, 03-(oleoyl)cholenic acid, dimethoxytrityl, or phenoxazine. Diverse lipid conjugates can preferentially drive oligonucleotide uptake into different tissues (Biscans et al, Nucleic Acids Res. 2019, 47, 1082-1096).

Additional conjugate groups include carbohydrates, phospholipids, antibodies, peptides, biotin, phenazine, folate, phenanthridine, anthraquinone, acridine, fluoresceins, rhodamines, coumarins, and dyes. In some embodiments, conjugation of a ligand to an antisense oligonucleotide allows recognition by cell-surface receptors (see, e.g., Wolfrum et al., Nat. Biotechnol. 2007, 25:1149-1157; Hostetler et al., Antiviral Chem. Chemother. 2001, 12:61-70; and Prakash et al., Nucleic Acids Res. 2014, 42:8796-807). In certain embodiments, the conjugate is a fibronectin type III (FN3) domain, such as a centyrin protein (see, e.g., Goldberg et al., Protein Eng Des Sel. 2016, 29(12):563-572). The various moieties, ligands, sequences, or conjugates of the disclosure and means to conjugate them to antisense compounds are described in further detail in WO2017/030973A1 and WO2018/031933A2, incorporated herein by reference.

Antisense oligonucleotides can also be modified to have one or more stabilizing groups that are generally attached to one or both termini of antisense oligonucleotides to enhance properties such as, for example, nuclease stability. Included in stabilizing groups are cap structures. These terminal modifications protect the antisense oligonucleotide having terminal nucleic acid from exonuclease degradation, and can help in delivery and/or localization within a cell. The cap can be present at the 5′-terminus (5′-cap), or at the 3′-terminus (3′-cap), or can be present on both termini. Cap structures include, for example, inverted deoxy abasic caps. Further 3′ and 5′-stabilizing groups that can be used to cap one or both ends of an antisense oligonucleotide to impart nuclease stability include those disclosed in WO 03/004602 published on Jan. 16, 2003.

In some embodiments, an antisense oligonucleotide of the present disclosure comprises a conjugate. In one embodiment, an antisense oligonucleotide of the present disclosure comprises a antisense oligonucleotide sequence and a conjugate, wherein the conjugate is linked to the antisense oligonucleotide sequence. In some embodiments, the conjugate is selected from any of the conjugates described herein, for example, a hydrophobic conjugate, a tissue-targeting conjugate, or a conjugate designed to optimize pharmacokinetic parameters. A hydrophobic conjugate useful for conjugating to antisense oligonucleotides of the present disclosure, includes a hexadecyloxypropyl conjugate, a cholesterol conjugate, a polyunsaturated fatty acid conjugate, and others known in the art that can improve cellular uptake of a conjugate antisense oligonucleotide. In some embodiments, the conjugate can be a tissue-targeting conjugate, for example, a carbohydrate conjugate, or a peptide conjugate, or any conjugate known in the art that can target an antisense oligonucleotide of the present disclosure to a specific tissue. In some embodiments, an antisense oligonucleotide of the present disclosure is conjugated with a polyethylene glycol conjugate. In one embodiment, a polyethylene glycol conjugate antisense oligonucleotide optimizes pharmacokinetic properties of the antisense oligonucleotide.

In some embodiments, the present disclosure provides biocleavable analogues of antisense oligonucleotides described herein. In such cases, biocleavable analogues comprise a hydrophobic conjugate that leads to stronger association with cell membranes and a linker. In one embodiment, the linker is a cleavable linker that when cleaved, releases the antisense oligonucleotide, e.g., releases the antisense oligonucleotide into endosomes. In some embodiments, an antisense compound comprises a cleavable linker, wherein the cleavable linker degrades when cleaved. In some embodiments, the linker is a nuclease-cleavable linker comprising a phosphodiester linkage. In some embodiments, the nuclease-cleavable linker comprising a phosphodiester linkage is about 2 to about 8 nucleotides in length. For example, a nuclease-cleavable phosphodiester linker can be 3, 4, 5, 6, 7, 8 nucleotides in length, or longer, e.g., 9, 10, 11, 12, 13, 14, 15, 16, 17, 18 nucleotides in length, or longer. In one embodiment, the nuclease-cleavable linker comprises about 6 nucleotides. In some embodiments, the cleavable linker is cleaved after cellular internalization. In some embodiments, the cleavable linker is cleaved within an endosome. In some embodiments, the cleavable linker is cleaved under reducing conditions. In some embodiments, the cleavable linker is cleaved under changing pH conditions, for example the cleavable linker is cleaved when the pH decreases, or when the pH increases. In some embodiments, the cleavable linker is cleaved by an intracellular nuclease or protease. In some embodiments, the cleavable linker is cleaved by an endosomal nuclease or protease.

### Pharmaceutical Compositions and Formulations

Provided herein are pharmaceutical compositions and formulations which include the antisense compounds described herein. For example, the antisense oligonucleotides described herein can be admixed, encapsulated, conjugated or otherwise associated with other molecules, molecule structures or mixtures of compounds. A pharmaceutical composition of the disclosure is formulated to be compatible with its intended route of administration. Examples of routes of central nervous system administration include intrathecal administration, intraventricular administration or intrastriatal administration. In some embodiments, the administration can employ an implanted device such as an Ommaya reservoir or implanted intrathecal catheter. Examples of systemic administration include intravenous administration, subcutaneous administration, or intramuscular administration. The route of administration will be, in part, dictated by the target tissue for the antisense compounds. Solutions or suspensions used for administration can include the following components: a sterile diluent such as water for injection, saline solution, lactated Ringers solution, Elliotts B solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates, carbonates or phosphates and agents for the adjustment of tonicity such as sodium chloride or dextrose. pH can be adjusted with acids or bases, such as hydrochloric acid or sodium hydroxide. The pharmaceutical compositions can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. In all cases, the composition must be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be suitable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, some methods of preparation are vacuum drying and freeze-drying which yields a powder of the active ingredient plus any additional desired ingredient from a previously sterile-filtered solution thereof.

The pharmaceutical compositions and formulations provided herein can, in some embodiments, be conveniently presented in unit dosage form and can be prepared according to conventional techniques. Such techniques can include bringing into association the active ingredients with the pharmaceutical carrier(s) or excipient(s). In general, the formulations can be prepared by uniformly and intimately bringing into association the active ingredients with liquid carriers, finely divided solid carriers, or both, and then, if necessary, shaping the product (e.g., into a specific particle size for delivery). In one embodiment, the pharmaceutical formulations are prepared for intrathecal, intraventricular or intrastriatal administration in an appropriate solvent, e.g., water or normal saline. In another embodiment, the pharmaceutical formulations are prepared for intravenous, subcutaneous or intramuscular administration in an appropriate solvent, e.g., water or normal saline.

An agent of the present disclosure, e.g., an antisense compound targeting a EXOC2, Ku80, and Task1 transcript can also be administered by transfection or infection using methods known in the art, including but not limited to the methods described in McCaffrey et al. (2002), Nature, 418(6893), 38-9 (hydrodynamic transfection); Xia et al. (2002), Nature Biotechnol., 20(10), 1006-10 (viral-mediated delivery); or Putnam (1996), Am. J. Health Syst. Pharm. 53(2), 151-160, erratum at Am. J. Health Syst. Pharm. 53(3), 325 (1996).

An agent of the present disclosure, e.g., an antisense compound targeting a EXOC2, Ku80, and Task1 transcript can also be administered by any method suitable for administration of nucleic acid agents, such as a DNA vaccine. These methods include gene guns, bio injectors, and skin patches as well as needle-free methods such as the micro-particle DNA vaccine technology disclosed in U.S. Pat. No. 6,194,389, and the mammalian transdermal needle-free vaccination with powder-form vaccine as disclosed in U.S. Pat. No. 6,168,587. Additionally, intranasal delivery is possible, as described in, inter alia, Hamajima et al. (1998), Clin. Immunol. Immunopathol., 88(2), 205-10. Liposomes (e.g., as described in U.S. Pat. No. 6,472,375) and microencapsulation can also be used. Biodegradable targetable microparticle delivery systems can also be used (e.g., as described in U.S. Pat. No. 6,471,996).

In one embodiment, the active agents are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

Toxicity and therapeutic efficacy of such compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds that exhibit large therapeutic indices are suitable. Although compounds that exhibit toxic side effects can be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies within a range of circulating concentrations that include the ED50 with little or no toxicity. The dosage can vary within this range depending upon the dosage form employed and the route of administration utilized. For any compound used in the method of the disclosure, the therapeutically effective dose can be estimated initially from cell culture assays. A dose can be formulated in animal models to achieve a circulating plasma concentration range that includes the EC50 (i.e., the concentration of the test compound which achieves a half-maximal response) as determined in cell culture. Such information can be used to more accurately determine useful doses in humans. Levels in plasma can be measured, for example, by high performance liquid chromatography.

The pharmaceutical compositions can be included in a container, pack, or dispenser together with instructions for administration.

An antisense compound targeted to a EXOC2, Ku80, and Task1 nucleic acid can be utilized in pharmaceutical compositions by combining the antisense compound with a suitable pharmaceutically acceptable diluent or carrier. A pharmaceutically acceptable diluent includes phosphate-buffered saline (PBS). PBS is a diluent suitable for use in compositions to be delivered parenterally. Accordingly, in one embodiment, employed in the methods described herein is a pharmaceutical composition comprising an antisense compound targeted to a EXOC2, Ku80, and Task1 nucleic acid and a pharmaceutically acceptable diluent. In certain embodiments, the pharmaceutically acceptable diluent is PBS. In certain embodiments, the antisense compound is an antisense oligonucleotide.

In certain embodiments, the pharmaceutically acceptable diluent is designed to mimic the composition of cerebrospinal fluid. As such, it can contain divalent salts such as Mg²⁺ and Ca²⁺. Elliotts B solution is a diluent suitable for use in compositions to be delivered into the cerebrospinal fluid. A person of skill in the art will be able to see that other buffer solutions, with variations in the concentrations of different monovalent and divalent ions, can also be suitable as pharmaceutically acceptable diluents.

Pharmaceutical compositions comprising antisense compounds encompass any pharmaceutically acceptable salts, esters, or salts of such esters, or any other oligonucleotide which, upon administration to an animal, including a human, is capable of providing (directly or indirectly) the biologically active metabolite or residue thereof. Accordingly, for example, the disclosure is also drawn to pharmaceutically acceptable salts of antisense compounds, prodrugs, pharmaceutically acceptable salts of such prodrugs, and other bioequivalents. Suitable pharmaceutically acceptable salts include, but are not limited to, sodium and potassium salts. A prodrug can include the incorporation of additional nucleosides at one or both ends of an antisense compound which are cleaved by endogenous nucleases within the body, to form the active antisense compound.

### Methods of Treatment

The present disclosure provides a method of treating a subject having a EXOC2, Ku80, or Task1-related disorder. Methods of treatment include administering to the subject in need thereof an effective amount of an antisense compound described herein. In certain embodiments, the antisense compound comprises a target-recognition sequence that is sufficiently complementary to a EXOC2, Ku80, or Task1 nucleic acid (e.g., a EXOC2, Ku80, or Task1 transcript) to direct cleavage of the EXOC2, Ku80, or Task1 nucleic acid by RNase H. In certain embodiments, the antisense compound comprises a target-recognition sequence that is sufficiently complementary to a EXOC2, Ku80, or Task1 nucleic acid (e.g., a EXOC2, Ku80, or Task1 transcript) to increase expression of the EXOC2, Ku80, or Task1 nucleic acid.

Methods of treating a subject having a EXOC2, Ku80, or Task1-related disorder are useful in treating any EXOC2, Ku80, or Task1related disorder known to those of ordinary skill in the art.

In another aspect, the disclosure provides a method of treating or managing amyotrophic lateral sclerosis (ALS) in a patient, comprising administering to the patient a therapeutically effective amount of an oligonucleotide with complementarity to SOD1 described herein.

In another aspect, the disclosure provides a method of treating or managing primary hyperlipidemia in a patient, comprising administering to the patient a therapeutically effective amount of the dsRNA targeting PCSK9 described herein.

In some embodiments, the primary hyperlipidemia is heterozygous familial hypercholesterolemia (HeFH).

In another aspect, the disclosure provides a method of reducing low density lipoprotein cholesterol (LDL-C) in a patient, comprising administering to the patient a therapeutically effective amount of the dsRNA targeting PCSK9 described herein.

The contents of the articles, patents, and patent applications, and all other documents and electronically available information mentioned or cited herein, are hereby incorporated by reference in their entirety to the same extent as if each individual publication was specifically and individually indicated to be incorporated by reference. Applicants reserve the right to physically incorporate into this application any and all materials and information from any such articles, patents, patent applications, or other physical and electronic documents.

While the present disclosure has been described with reference to the specific embodiments thereof, it should be understood by those skilled in the art that various changes can be made and equivalents can be substituted without departing from the true spirit and scope of the disclosure. It will be readily apparent to those skilled in the art that other suitable modifications and adaptations of the methods described herein can be made using suitable equivalents without departing from the scope of the embodiments disclosed herein. In addition, many modifications can be made to adapt a particular situation, material, composition of matter, process, process step or steps, to the objective, spirit and scope of the present disclosure. All such modifications are intended to be within the scope of the claims appended hereto. Having now described certain embodiments in detail, the same will be more clearly understood by reference to the following examples, which are included for purposes of illustration only and are not intended to be limiting.

### Examples

The present disclosure is further illustrated by the following examples which should not be construed as further limiting.

### Example 1—Materials and Methods

iPSC Culture

iPSC lines from a C9ORF72 carrier and its isogenic control were characterized before (Lopez-Gonzalez et al., 2019). iPSCs were cultured (Lopez-Gonzalez et al., 2019). Briefly, iPSCs were maintained in Matrigel ((Cat #354230, Corning), diluted 1:100 in KnockOut DMEM/F-12 (Cat #12660012, Gibco)) coated 6-well plates in mTeSR1 media (mTeSR1 Basal Media with mTeSR1 5X supplement, Stem Cell Technologies). The mTeSR1 media were replaced every day. iPSCs were passaged every 4-6 days. Cells were washed with DPBS, dissociated in Accutase ((Cat #SCR005, Millipore) diluted 1:2 in DPBS) for ˜1 min at room temperature (RT), washed with DPBS, and scraped in fresh mTeSR1 media using cell lifter. After reaching the desired colony size, cells were seeded in Matrigel coated 6-well plates in fresh mTeSR1 media. Spontaneously differentiated colonies were manually removed prior to splitting and motor neuron differentiation.

### Motor Neuron Differentiation

iPSCs were differentiated into spinal motor neurons according to a protocol from Du et al., 2015 with some modifications (Du et al., 2015; Lopez-Gonzalez et al., 2019). iPSCs with small colony size were seeded in Matrigel coated 6-well plates ˜50% confluency in mTeSR1 media. Next day, media were changed to neuroepithelial progenitor (NEP) induction media, which is neural media KnockOut DMEM/F-12 medium (Cat #12660012, Gibco) and Neurobasal medium (Cat #21103049, Gibco) at 1:1, 0.5X N2 Supplement (Cat #17502-048, Gibco), 0.5X B27 Supplement (Cat #17504044, Gibco), 0.1 mM ascorbic acid (Cat #A4403, Sigma), 1X Glutamax (Cat #35050061, Thermo Fisher Scientific), supplemented with 3 μM CHIR99021 (Cat #72054 Stem Cell Technologies), 2 μM DMH1 (Cat #4126 Tocris Bioscience), and 2 μM SB431542 (Cat #04-0010-10, Stemgent). NEP media were changed every other day for 6 days. NEPs were dissociated with 1:2 diluted Accutase and split 1:3 into Matrigel-coated 6-well plates. NEPs were cultured in motor neuron progenitor (MNP) induction media, the neural media supplemented with 1 μM CHIR99021, 2 μM DMH1, 2 μM SB431542, 0.1 μM Retinoic acid (Cat #R2625, Sigma) and 0.5 μM Purmorphamine (Cat #540220, Calbiochem). MNP media were changed every other day for 6 days. MNPs were dissociated with 1:2 diluted Accutase and cultured in suspension in 60 mm low attachment plates in motor neuron differentiation media, the neural media supplemented with 0.5 μM Retinoic acid and 0.1 μM Purmorphamine) for 6 days. The media were changed every other day for 6 days. Lastly, neurospheres were dissociated into single cells with Accutase (for 10 min, at 37° C.), passed through 40 μm filter into motor neuron media, the neural media supplemented with 0.5 μM Retinoic acid, 0.1 μM Purmorphamine, 0.1 μM Compound E (Cat #73954, Stem Cell Technologies), 10 ng/ml BDNF (Cat #248-BDB, R&D Systems), 10 ng/ml GDNF (Cat #PHC7041, Thermo Fisher Scientific), and 1 μg/ml Laminin (Cat #L2020, Sigma). Cells were seeded in Matrigel coated 12-well plates at 5×10⁵ cells/well density in motor neuron media. After two weeks, neurons were maintained in motor neuron media without Retinoic acid and Purmorphamine. The media were half-changed once a week for three weeks.

Cell culture

HEK293T cell lines were purchased from the American Type Culture Collection (ATCC, Cat #CRL-1 1268). Wild type (Cat #ab255448) and Larnp2 knockout (Cat #ab255402) cell lines were purchased from Abcam. Cells were maintained in Dulbecco's Modified Eagle's Medium (DMEM: Cat #10--013-CV, Corning) supplemented with 10% Fetal Bovine Serum (FBS; Cat #10-437-028, Thermo Fisher Scientific) and 1% Penicillin/Streptomycin, Cells were seeded in 12-well plates at 2×10⁵ cells/well density for treatments.

### ASO Treatment

Three weeks old iPSC-derived neurons or cell lines were treated with ASOs for two days at the indicated concentrations. ASOs were directly added to culture media for free uptake. Cells were harvested two days after ASO treatment for RNA extraction. List of ASOs is shown in Table 1.

Nontargeting control (NTC), EXOC2-349. EXOC2-393. Ku80-624, Ku80-1425, Ku80-2802. Task1-692, Task1-5968 ASOs were designed by IDT Custom Design Service. All ASOs listed in Table 1 were purchased from IDT.

### Bafilomycin A1 Treatment

One day after seeding, HEK293T cells were treated with 50 nM Bafilomycin A1 (BafA1) or DMSO for 24 hrs. Next day, cells were treated with ASOs and 10 nM BafA1. Cells were harvested two days after ASO treatment for RNA extraction.

### RNA Extraction and Real-Time Quantitative PCR

Total RNA from iPSC-MNs was extracted with RNeasy Mini kit (Cat #74106, Qiagen). 1 μg RNA was reverse transcribed to cDNA using random hexamers with TaqMan Reverse Transcription kit (Cat #N8080234, Thermo Fisher Scientific). Real-time quantitative PCR was done using SYBR Select Master Mix (Cat #4472918, Thermo Fisher Scientific) using Applied Biosystems Quant Studio 3 System. Ct values for each sample were normalized to cyclophilin. Relative mRNA levels of each gene were calculated using 2^{-ΔΔct} method. qRT-PCR primers are listed in Table 2.

### Example 2—EXOC2 mRNA Level as Measured by qRT-PCT Analysis for Gapmers with and without Poly-G, Poly-A, or Poly-C Ligand

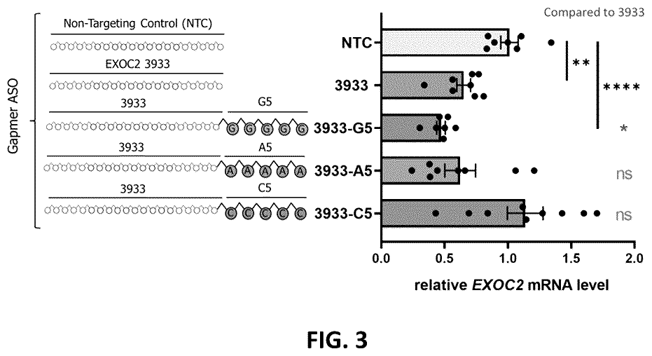

The relative EXOC2 mRNA level as measured by qRT-PCT analysis for gapmers with and without poly-G, poly-A, or poly-C ligand was assessed (FIG. 3).

The qRT-PCR analysis of the relative EXOC2 mRNA levels in HEK cells treated with 5 uM Nontargeting control (NTC) or EXOC2 targeting Gapmer ASO 3933, LysoASO 3933-G5, or ASOs with other additional sequences 3933-A5 or 3933-C5 for two days was performed.

ASO sequence with poly-G ligand was found to be specific and provide additional knockdown compared to ASO without poly-G ligand. ASO 3933 was found to significantly reduce EXOC2 mRNA levels compared to NTC ASO. The addition of Lyso sequence (GGGGG) to 3933, 3933-G5, was found to significantly increased knockdown efficiency of Gapmer ASO 3933. Enhancement in knockdown efficiency was specific to Lyso sequence since addition of (AAAAA) or (CCCCC) to 3933 did not significantly change the relative EXOC2 mRNA level compared to 3933.

### Example 3—EXOC2 mRNA Level as Measured by qRT-PCT Analysis for Gapmers with and without Poly-G Ligand of Different Lengths

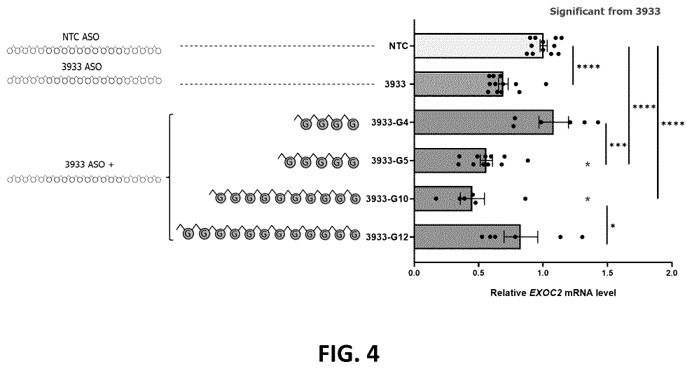

The relative EXOC2 mRNA level as measured by qRT-PCT analysis for gapmers with and without poly-G ligand of different lengths was assessed (FIG. 4).

The qRT-PCR analysis of relative EXOC2 mRNA levels in HEK cells treated with 5 uM NTC, EXOC2 targeting Gapmer ASO 3933, and LysoASOs with different lengths of Gs 3933-G4, 3933-G5, 3933-G10, and 3933-G12 for two days was performed.

The ASO sequence with poly-G ligand was found to have a range of 5-10 Gs. ASO 3933 was found to significantly reduced EXOC2 mRNA levels compared to NTC ASO. ASOs 3933-G5 and 3933-G10 were found to significantly increase knockdown efficiency of Gapmer ASO 3933. ASOs 3933-G4 and 3933-G12 were found to do not lower EXOC2 mRNA levels.

### Example 4—EXOC2 mRNA Level as Measured by qRT-PCT Analysis for EXOC2 349

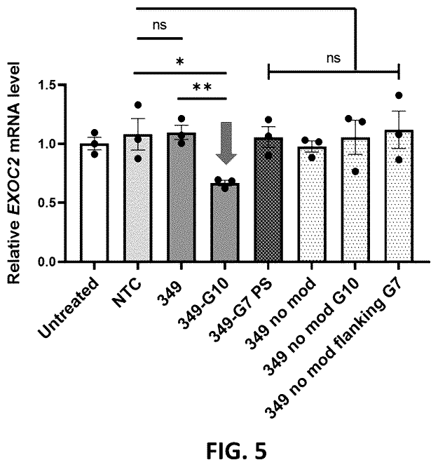

The relative EXOC2 mRNA level as measured by qRT-PCT analysis for EXOC2 349 was assessed (FIG. 5).

The qRT-PCR analysis of relative EXOC2 mRNA levels in iPSC-derived neurons treated with 5 uM NTC, EXOC2 targeting Gapmer ASO 349, LysoASOs 349-G10, 349-G7 PS, and other oligos without modifications 349 no mod, 349 no mod G10, 349 no mod flanking G7 for two days was performed.

The ASO with poly-G ligand was found to render non-working ASO functional in iPSC-derived neurons. Gapmer ASO 349 was found to do not change the relative EXOC2 mRNA level compared to NTC ASO. ASO 349-G10 was found to significantly reduce EXOC2 mRNA levels compared to NTC or ASO 349. ASO 349-G7 PS was found to do not change the relative EXOC2 mRNA level compared to NTC ASO suggesting that full or partial PS modification might prevent recognition of Lyso sequence by Lamp2C receptor.

### Example 5—EXOC2 mRNA Level as Measured by qRT-PCT Analysis for EXOC2 349

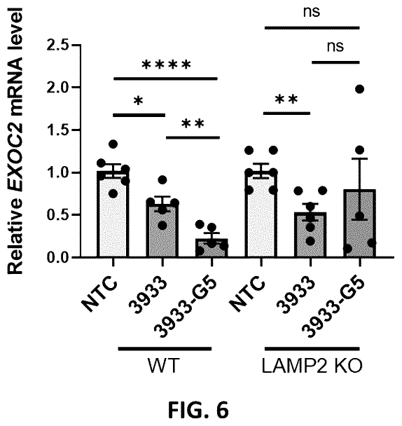

The relative EXOC2 mRNA level as measured by qRT-PCT analysis for EXOC2 3933 was assessed (FIG. 6).

The qRT-PCR analysis of relative EXOC2 mRNA levels in wildtype (WT) and LAMP2 knockout (KO) HeLa cells treated with 5 uM NTC, EXOC2 targeting Gapmer ASO 3933 and LysoASOs 3933-G5 for two days. Gapmer ASO 3933 significantly reduced the relative EXOC2 mRNA level in WT and LAMP2 KO cells compared to NTC ASO was performed.

The effect of LysoASO was found to be mediated via LAMP2 receptor. LysoASO 3933-G5 was fond to further increase the knockdown efficiency of ASO 3933. EXOC2 mRNA levels were found to be significantly reduced in LysoASO 3933-G5 group compared to ASO 3933 group in WT cells. However, this effect was not observed in LAMP2 KO cells, LysoASO 3933-G5 did not change EXOC2 mRNA levels compared to NTC or ASO 3933.

### Example 6—EXOC2 mRNA Level as Measured by qRT-PCT Analysis for EXOC2 3933 with inhibited and un-inhibited lysosome

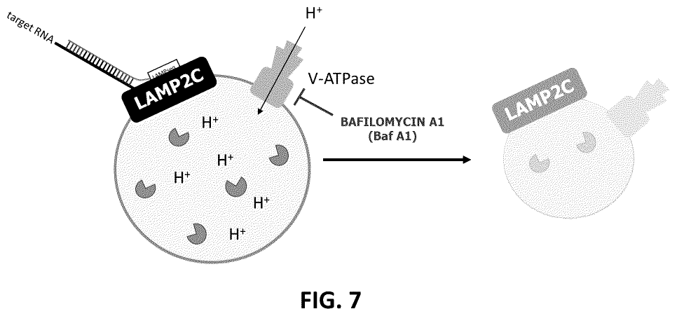

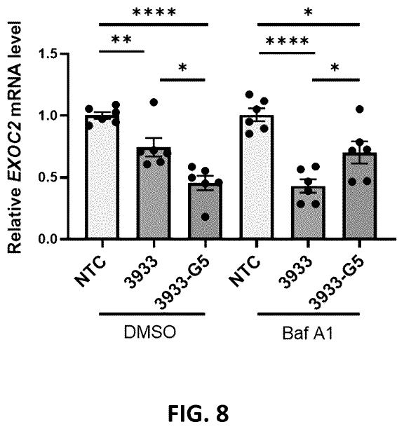

The relative EXOC2 mRNA level as measured by qRT-PCT analysis for EXOC2 3933 with inhibited and un-inhibited lysosome was assessed (FIG. 7 and FIG. 8).

The qRT-PCR analysis of relative EXOC2 mRNA levels in HEK cells treated with 5 uM NTC, EXOC2 targeting Gapmer ASO 3933, and LysoASO 3933-G5 for two days was performed. HEK cells were treated with DMSO or 50 nM Bafilomycin A1 (Baf A1), lysosome inhibitor, for 24 hrs. prior to ASO treatment.

LysoASO was found to mediates its effect via functional lysosomes. Gapmer ASO 3933 was found to significantly reduce the relative EXOC2 mRNA level in DMSO and Baf A1 treated cells compared to NTC ASO. LysoASO 3933-G5 was found to further increase the knockdown efficiency of ASO 3933. EXOC2 mRNA levels were fond to be significantly reduced in LysoASO 3933-G5 group compared to ASO 3933 group in DMSO treated cells. However, this effect was abrogated in Baf A1 treated cells, LysoASO 3933-G5 did not further reduce EXOC2 mRNA levels compared to ASO 3933.

### Example 7—EXOC2 mRNA Level as Measured by qRT-PCT Analysis for Sterically Blocked and not Blocked EXOC2 3933 that is

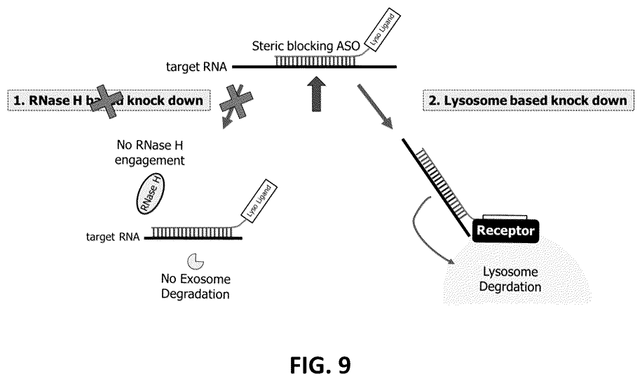

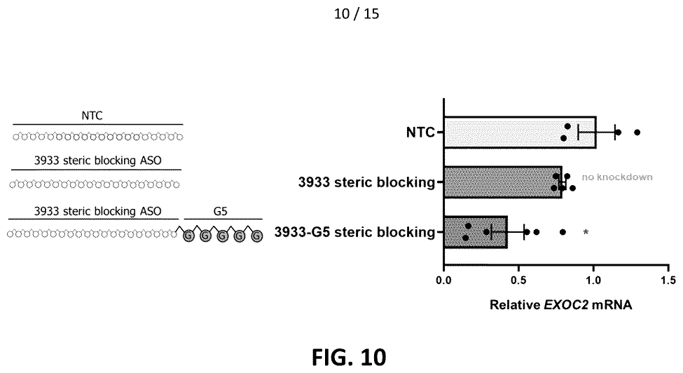

The relative EXOC2 mRNA level as measured by qRT-PCT analysis for sterically blocked and not blocked EXOC2 3933 was assessed (FIG. 9 and FIG. 10).

The qRT-PCR analysis of relative EXOC2 mRNA levels in HEK cells treated with 5 uM NTC, EXOC2 targeting steric blocking ASO 3933, and LysoASO 3933-G5 steric blocking ASO for two days was performed.

LysoASO was fond to target RNA to RNase H independent pathway for degradation. Expectedly, steric blocking ASO 3933 was found to do not change the relative EXOC2 mRNA level compared to NTC. LysoASO steric blocking ASO 3933-G5 however, was found to significantly reduce the level of EXOC2 mRNA compared to NTC suggesting engagement of a degradation pathway alternative to RNase H.

### Example 8—Ku80 mRNA Level as Measured by qRT-PCT Analysis for Ku80 624 and 2802

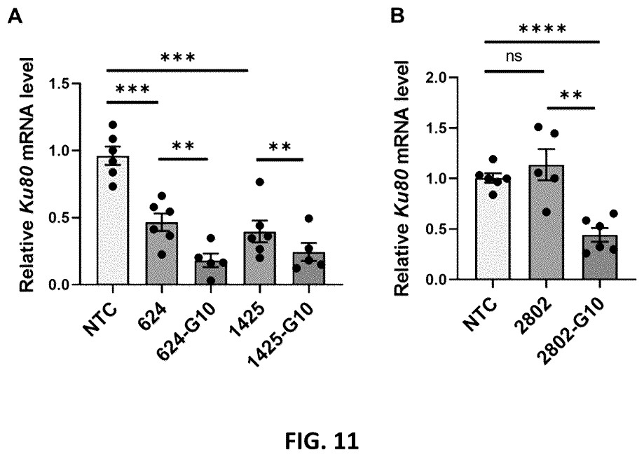

The relative Ku80 mRNA level as measured by qRT-PCT analysis for Ku80 624 and 2802 was assessed (FIG. 11).

The qRT-PCR analysis of relative Ku80 mRNA levels in (A) iPSC-derived neurons treated with 1 uM NTC, Ku80 targeting Gapmer ASOs 624 and 1425, LysoASOs 624-G10 and 1425-G10, in (B) HeLa cells treated with 1 uM NTC, Ku80 targeting Gapmer ASO 2802 and LysoASO 2802-G10 for two days was performed.

LysoASO was found to effectively induce Ku80 mRNA for degradation. Gapmer ASOs 624 and 1424 were found to significantly reduce Ku80 mRNA levels compared to NTC. LysoASOs 624-G10 and 1425-G10 were found to further reduce the level of Ku80 mRNA compared to Gapmer ASOs 624 and 1424 respectively. Gapmer ASO 2802 was found to do not change Ku80 mRNA level compared to NTC. LysoASO 2802-G10 was found to significantly reduce the level of Ku80 mRNA compared to NTC and ASO 2802.

### Example 9—TASK1 mRNA Level as Measured by qRT-PCT Analysis for TASK1 622 and 5968

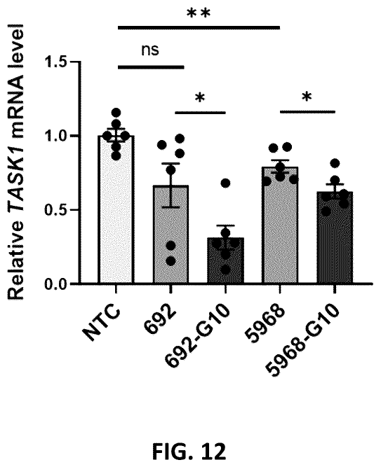

The relative TASK1 mRNA level as measured by qRT-PCT analysis for TASK1 622 and 5968 was assessed (FIG. 12).

The qRT-PCR analysis of relative Task1 mRNA levels in iPSC-derived neurons treated with 5 uM NTC, Task1 targeting Gapmer ASOs 692 and 5968, LysoASOs 692-G10 and 5968-G10 for two days was performed.

LysoASO was found to effectively induces Task1 mRNA for degradation. ASO 692 was found to do not change Task1 mRNA level compared to NTC, and ASOs 5968 was found to significantly reduce Task1 mRNA levels compared to NTC. LysoASOs 692-G10 and 5968-G10 were found to further reduce the level of Task1 mRNA compared to ASOs 692 and 5968 respectively and rendered nonworking ASO 692 working.

### Example 10—Enhancement of Silencing from Approved Oligonucleotide Therapeutics with a Poly-G Ligand

An advantage of the poly-G ligand described herein is the broad utility to enhance silencing in any therapeutic oligonucleotide, including approved oligonucleotide therapeutics that have already been selected to robust silencing efficacy. To demonstrate this utility, two approved oligonucleotide therapeutics were tested with and without the poly-G ligand, the anti-SOD1 ASO tofersen (QALSODY®), and the anti-PCSK9 siRNA inclisiran (LEQVIO®).

### Example 10—Materials and Methods

### Cell Culture

SH-SY5Y cells were maintained in DMEM/F-12 (Dulbecco's Modified Eagle Medium/Nutrient Mixture F-12) supplemented with 10% FBS (Fetal Bovine Serum) and 1% Penicillin/Streptomycin (Pen-strep). A549 cells were maintained in F-12 medium supplemented with 10% FBS and 1% Pen-strep. Cells were seeded in 24-well plates at 2×10⁵ cells/well density for treatments.

### ASO Treatment

SH-SY5Y cells were transfected with 25 or 100 nM ASOs, listed in Table 3, using Lipofectamine RNAiMAX. Cells were harvested 24 hrs. after the ASO treatment for RNA extraction and RT-qPCR analysis.

siRNA Treatment

Sense siRNA was hybridized with either antisense strand or Lamp-antisense strand, A549 cells were transfected with 50 nM siRNAs, listed in Table 4, using Lipofectamine RNAiMAX Cells were harvested 48 hrs. after the siRNA treatment for RNA extraction and RT-qPCR analysis.

### RNA Extraction and Real-Time Quantitative PCR

Total RNA was using acid guanidinium thiocyanate phenol-chloroform extraction (QIAzol reagent, Qiagen) following the manufacturer protocol. 1 μg RNA was reverse transcribed to cDNA using random hexamers and ProtoScript II reverse transcriptase (New England Biolabs). Real-time quantitative PCR (RT-qPCR) was done using a Bio-rad CFX96 real-time PCR instrument. Ct values for each sample were normalized to GAPDH. Relative mRNA levels of each gene were calculated using 2^{-ΔΔCt} method. qRT-PCR primers are listed in Table 5.

### Example 10—Results

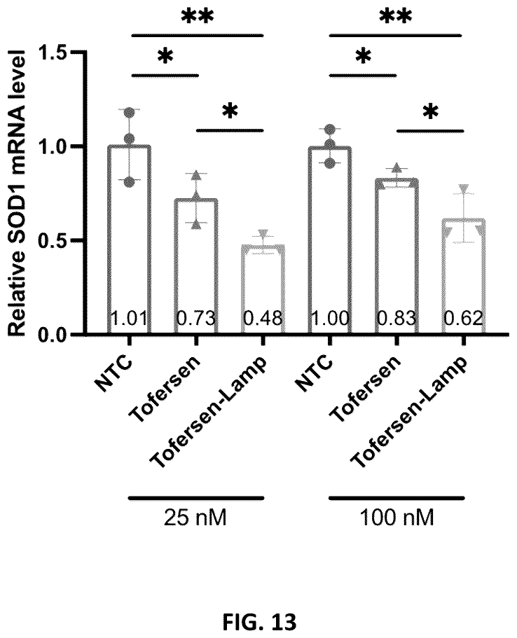

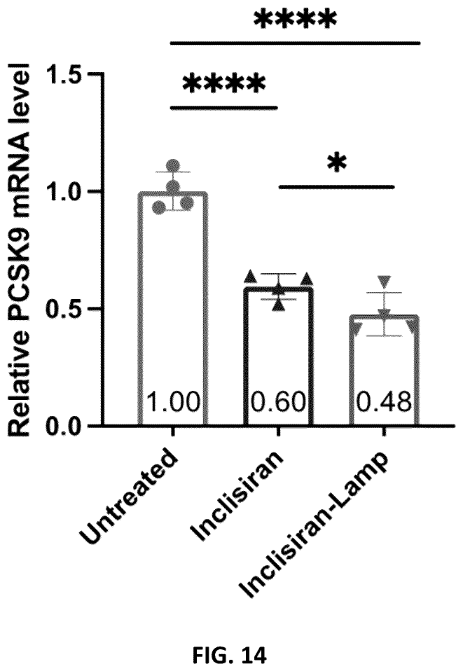

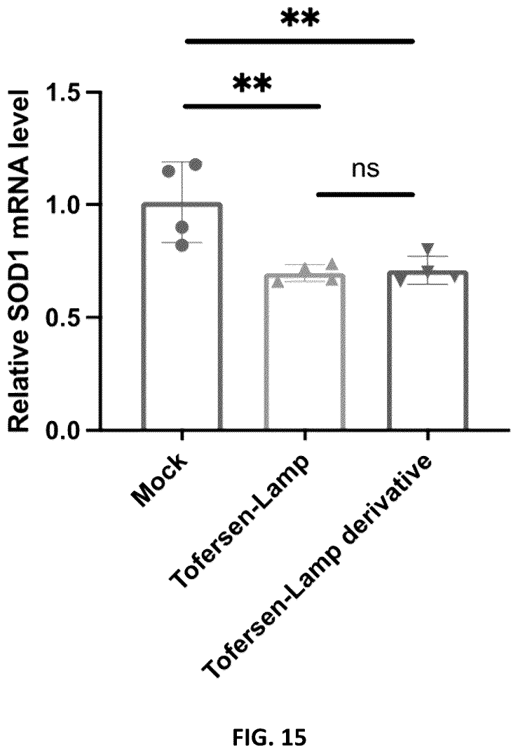

The ASO tofersen was tested with and without the poly-G ligand 10G (SEQ ID NO: 56)(i.e., a Lamp ligand) in SH-SY5Y cells treated with 25 or 100 nM of each compound. As shown in FIG. 13, the silencing efficacy of tofersen was markedly improved with the inclusion of the 10G ligand, based on relative SOD1 expression compared to a non-target control. An alternative poly-G ligand was also tested with tofersen (tofersen-Lamp derivative). This alternative poly-G ligand does not contain consecutive G nucleotides, rather an intervening non-G nucleotide (A nucleotide) is present after every 3 G nucleotides. As shown in FIG. 15, the alternative poly-G ligand was equally effective at enhancing SOD1 repression as the consecutive G ligand 10G (SEQ ID NO: 56).

The siRNA inclisiran was also tested with and without a poly-G ligand. In this instance, a 5G ligand was employed at the 3′ end of the antisense strand. A549 cells treated with 50 nM inclisiran or inclisiran-Lamp for 48 hrs. As shown in FIG. 14, the silencing efficacy of inclisiran was also markedly improved with the inclusion of a poly-G ligand (the 5G ligand), based on relative PCSK9 expression compared to a non-target control.

Importantly, both of these figures demonstrate the utility of the poly-G ligand for enhancing silencing in two different therapeutic oligonucleotide types, ASOs and siRNAs. Moreover, the poly-G ligand enhanced silencing in therapeutic oligonucleotide comprising a wide variety of chemical modifications.

This work demonstrates that the poly-G ligand is broadly applicable to any oligonucleotide type, regardless of the underlying oligonucleotide sequence or chemical modification usage.

Sequences

TABLE 1
ASOs.

| SEQ | | |
|---|---|---|
| ID | | |
| NO | ASO | Sequence (5′ > 3′) |
| 1 | Nontargeting | G*C*G*A*C*T*A*T*A*C* |
| | control | G*C*G*C*A*A*T*A*T*G |
| | (NTC) | |
| 2 | EXOC2-349 | T*T*T*T*G*G*C*T*T*G* |
| | | T*C*C*C*A*C*T*C*G*A |
| 3 | EXOC2-349- | T*T*T*T*G*G*C*T*T*G* |
| | G10 | T*C*C*C*A*C*T*C*G*A* |
| | | G-G-G-G-G-G-G-G-G-G |
| 4 | EXOC2-349- | T*T*T*T*G*G*C*T*T*G* |
| | G7 PS | T*C*C*C*A*C*T*C*G*A* |
| | | G*G*G*G*G*G*G |
| 5 | EXOC2-349 | T-T-T-T-G-G-C-T-T-G- |
| | no mod | T-C-C-C-A-C-T-C-G-A |
| 6 | EXOC2-349 | T-T-T-T-G-G-C-T-T-G- |
| | no mod | T-C-C-C-A-C-T-C-G-A- |
| | G10 | G-G-G-G-G-G-G-G-G-G |
| 7 | EXOC2-349 | G-G-G-G-G-G-G-T-T-T- |
| | flanking | T-G-G-C-T-T-G-T-C-C- |
| | G7 | C-A-C-T-C-G-A-G-G-G- |
| | | G-G-G-G |
| 8 | EXOC2- | A*A*G*T*G*C*A*T*G*C* |
| | 3933 | G*G*C*T*G*G*T*C*T*A |
| 9 | EXOC2- | A*A*G*T*G*C*A*T*G*C* |
| | 3933-A5 | G*G*C*T*G*G*T*C*T*A* |
| | | A-A-A-A-A |
| 10 | EXOC2- | A*A*G*T*G*C*A*T*G*C* |
| | 3933-C5 | G*G*C*T*G*G*T*C*T*A* |
| | | C-C-C-C-C |
| 11 | EXOC2-3 | A*A*G*T*G*C*A*T*G*C* |
| | 933-G5 | G*G*C*T*G*G*T*C*T*A* |
| | | G-G-G-G-G |
| 12 | EXOC2- | A*A*G*T*G*C*A*T*G*C* |
| | 3933-G4 | G*G*C*T*G*G*T*C*T*A* |
| | | G-G-G-G |
| 13 | EXOC2- | A*A*G*T*G*C*A*T*G*C* |
| | 3933-G10 | G*G*C*T*G*G*T*C*T*A* |
| | | G-G-G-G-G-G-G-G-G-G |
| 14 | EXOC2- | A*A*G*T*G*C*A*T*G*C* |
| | 3933-G12 | G*G*C*T*G*G*T*C*T*A* |
| | | G-G-G-G-G-G-G-G-G-G- |
| | | G-G |
| 15 | EXOC2- | A*A*G*T*G*C*A*T*G*C* |
| | 3933 | G*G*C*T*G*G*T*C*T*A |
| | steric | |
| | blocking | |
| 16 | EXOC2- | A*A*G*T*G*C*A*T*G*C* |
| | 3933-G5 | G*G*C*T*G*G*T*C*T*A* |
| | steric | G-G-G-G-G |
| | blocking | |
| 17 | Ku80- | A*A*G*T*G*A*G*T*C*C* |
| | 624 | G*T*C*C*T*T*A*C*C*C |
| 18 | Ku80- | A*A*G*T*G*A*G*T*C*C* |
| | 624-G10 | G*T*C*C*T*T*A*C*C*C* |
| | | G-G-G-G-G-G-G-G-G-G |
| 19 | Ku80- | A*T*T*C*A*A*C*T*G*T* |
| | 1425 | G*C*C*T*C*G*G*T*G*G |
| 20 | Ku80- | A*T*T*C*A*A*C*T*G*T* |
| | 1425-G10 | G*C*C*T*C*G*G*T*G*G* |
| | | G-G-G-G-G-G-G-G-G-G |
| 21 | Ku80- | A*A*G*T*G*A*G*T*C*C* |
| | 2802 | G*T*C*C*T*T*A*C*C*C |
| 22 | Ku80- | A*A*G*T*G*A*G*T*C*C* |
| | 2802-G10 | G*T*C*C*T*T*A*C*C*C* |
| | | G-G-G-G-G-G-G-G-G-G |
| 23 | Task1- | G*A*A*G*G*T*C*C*A*G* |
| | 692 | T*G*C*T*C*G*T*A*G*T |
| 24 | Task1- | G*A*A*G*G*T*C*C*A*G* |
| | 692-G10 | T*G*C*T*C*G*T*A*G*T* |
| | | G-G-G-G-G-G-G-G-G-G |
| 25 | Task1- | T*G*A*T*G*C*T*C*A*C* |
| | 5968 | C*C*C*G*A*G*T*A*A*C |
| 26 | Task1- | T*G*A*T*G*C*T*C*A*C* |
| | 5968-G10 | C*C*C*G*A*G*T*A*A*C* |
| | | G-G-G-G-G-G-G-G-G-G |

Note:
(-) indicates phosphodiester backbone,
(*) indicates phosphorothioate backbone.
(N) in black indicates nucleotides with no modification
and (N) in bold italics indicates nucleotides with 2′-O-methoxy-ethyl (2′MOE) sugar modification.

TABLE 2
qRT-PCR primers.

| SEQ | | |
|---|---|---|
| ID | | |
| NO | Primer | Sequence (5′ > 3′) |
| 27 | EXOC2 F | ACTGAAGGGGGTTCTGGAGT |
| 28 | EXOC2 R | CCAAACAAGTCAGGGGAAGA |
| 29 | Cyclophilin | TGCCATCGCCAAGGAGTAG |
| | F (Yuva- | |
| | Aydemir et | |
| | al., 2019) | |
| 30 | Cyclophilin | TGCACAGACGGTCACTCAAA |
| | R (Yuva- | |
| | Aydemir et | |
| | al., 2019) | |
| 31 | Ku80 F | CCCCAATTCAGCAGCATATT |
| | (Capraro et | |
| | al., 2011) | |
| 32 | Ku80 R | CCTTCAGCCAGACTGGAGAC |
| | (Capraro et | |
| | al., 2011) | |
| 33 | Task1 F | GGTGCTCATCGGCTTCTTCT |
| 34 | Task1 R | GAAGCTGAAGGCCACGTACT |

TABLE 3
ASO sequences. (#) indicates phosphorothioate
linkage. (eN) indicates 2′MOE, (dN) indicates
standard 2′ deoxyribonucleosides, and (d5N)
indicates 5-methyl-dN (e.g., 5-methyl
cytosine).

| SEQ | | |
|---|---|---|
| ID | | |
| NO | ASO | Sequence (5′ to 3′) |
| 35 | Nontargeting | (eG)#(eC)#(eG)#(eA)#(eC)#(dT)# |
| | control | (dA)#(dT)#(dA)#(dC)#(dG)#(dC)# |
| | (NTC) | (dG)#(dC)#(dA)#(eA)#(eT)#(eA)# |
| | | (eT)#(eG) |
| 36 | Tofersen | (eC)#(eA)(eG)#(eG)(eA)#(dT)# |
| | | (dA)#(d5C)#(dA)#(dT)#(dT)# |
| | | (dT)#(d5C)#(dT)#(dA)#(eC) |
| | | (eA)#(eG)(eC)#(eU) |
| 37 | Tofersen- | (eC)#(eA)(eG)#(eG)(eA)#(dT)# |
| | Lamp | (dA)#(d5C)#(dA)#(dT)#(dT)# |
| | | (dT)#(d5C)#(dT)#(dA)#(eC) |
| | | (eA)#(eG)(eC)#(eU) |
| | | (dG)(dG)(dG)(dG)(dG)(dG)(dG) |
| 38 | Tofersen- | (dG)(dG)(dG)(eC)#(eA)(eG)# |
| | Lamp | (eG)(eA)#(dT)#(dA)#(d5C)# |
| | derivative | (dA)#(dT)#(dT)#(dT)#(d5C)# |
| | | (dT)#(dA)#(eC)(eA)#(eG) |
| | | (eC)#(eU) |
| | | (dG)(dG)(dG)(dA)(dG)(dG) |
| | | (dG)(dA)(dG) |

TABLE 4
siRNA sequences. (#) indicates phosphorothioate
linkage. (mN) indicates 2′-OMe, (fN) indicates
2′-Fluoro nucleoside modifications, V indicates
5′-Vinyl phosphate.

| SEQ | | |
|---|---|---|
| ID | | |
| NO | siRNA | Sequence (5′ to 3′) |
| 39 | Inclisiran | (mC)#(mU)#(mA)(mG)(mA)(mC) |
| | Sense | (fC)(mU)(fG)(mU)(dT)(mU) |
| | Strand | (mU)(mG)(mC)(mU)(mU)(mU) |
| | | (mU)(mG)(mU)GalNac |
| 40 | Inclisiran | V(mA)#(fC)#(mA)(fA)(fA) |
| | Antisense | (fA)(mG)(fC)(mA)(fA)(mA) |
| | Strand | (mA)(mC)(fA)(mG)(fG)(mU) |
| | | (fC)(mU)(mA)(mG)#(mA)# |
| | | (mA) |
| 41 | Inclisiran- | V(mA)#(fC)#(mA)(fA)(fA) |
| | Lamp | (fA)(mG)(fC)(mA)(fA)(mA) |
| | Antisense | (mA)(mC)(fA)(mG)(fG)(mU) |
| | Strand | (fC)(mU)(mA)(mG)#(mA)# |
| | | (mA)(dG)(dG)(dG)(dG)(dG) |

TABLE 5
qRT-PCR primers.

| SEQ ID NO | Primer | Sequence (5′ to 3′) |
|---|---|---|
| 42 | SOD1 F | ACTGGTGGTCCATGAAAAAGC |
| 43 | SOD1I R | AACGACTTCCAGCGTTTCCT |
| 44 | PCSK9 F | CCTTCTTCCTGGCTTCCTGG |
| 45 | PCSK9 R | TCACAAAAGCCGAGGTAGCC |
| 46 | GAPDH F | TGCACCACCAACTGCTTA |
| 47 | GAPDH R | GGATGCAGGGATGATGTTC |

### REFERENCES

Aboul-Fadl, T. (2018). Antisense Oligonucleotides: Strategies, Designs, and Principles. Methods in Molecular Biology, 1768, 3-23.
Bennett, C. F., & Swayze, E. E. (2010). RNA Targeting Therapeutics: Molecular Mechanisms of Antisense Oligonucleotides as a Therapeutic Platform. Annual Review of Pharmacology and Toxicology, 50, 259-293.
Brummelkamp, T. R., Bernards, R., & Agami, R. (2002). A System for Stable Expression of Short Interfering RNAs in Mammalian Cells. Science, 296(5567), 550-553.
Boudreau, R. L., Spengler, R. M., Hylock, R. H., Kusenda, B. J., Davis, H. A., Eichmann, D. A., . . . & Davidson, B. L. (2011). siSPOTR: A Tool for Designing Highly Specific and Potent siRNAs for Human and Mouse. Nucleic Acids Research, 39(12), e82.
Corey, D. R. (2017). Nusinersen, an Antisense Oligonucleotide Drug for Spinal Muscular Atrophy. Nature Neuroscience, 20(4), 497-499.
Crooke, S. T., Witztum, J. L., Bennett, C. F., & Baker, B. F. (2018). RNA-Targeted Therapeutics. Cell Metabolism, 27(4), 714-739.
Daka, A., Peer, D., & Rothschild, G. (2020). Understanding Intracellular Barriers to mRNA-Based Therapeutics: A Panoramic Overview. Molecular Therapy, 28(4), 856-877.
Elbashir, S. M., Harborth, J., Lendeckel, W., Yalcin, A., Weber, K., & Tuschl, T. (2001). Duplexes of 21-Nucleotide RNAs Mediate RNA Interference in Cultured Mammalian Cells. Nature, 411(6836), 494-498.
Fitzgerald, K., Frank-Kamenetsky, M., Shulga-Morskaya, S., Liebow, A., Bettencourt, B. R., Sutherland, J. E., . . . & Akinc, A. (2021). Effect of an RNA Interference Drug on the Synthesis of Proprotein Convertase Subtilisin/Kexin Type 9 (PCSK9) and the Concentration of Serum LDL Cholesterol in Healthy Volunteers: A Randomized, Single-Blind, Placebo-Controlled, Phase 1 Trial. The Lancet, 383(9939), 60-68.
Fire, A., Xu, S., Montgomery, M. K., Kostas, S. A., Driver, S. E., & Mello, C. C. (1998). Potent and Specific Genetic Interference by Double-Stranded RNA in Caenorhabditis Elegans. Nature, 391(6669), 806-811.
Gilleron, J., Querbes, W., Zeigerer, A., Borodovsky, A., Marsico, G., Schubert, U., . . . & Akinc, A. (2013). Image-Based Analysis of Lipid Nanoparticle-Mediated siRNA Delivery, Intracellular Trafficking, and Endosomal Escape. Nature Biotechnology, 31(7), 638-646.
Hannon, G. J. (2002). RNA Interference. Nature, 418(6894), 244-251.
Jackson, A. L., & Linsley, P. S. (2010). Recognizing and Avoiding siRNA Off-Target Effects for Target Identification and Therapeutic Application. Nature Reviews Drug Discovery, 9(1), 57-67.
Juliano, R. L. (2016). The Delivery of Therapeutic Oligonucleotides. Nucleic Acids Research, 44(14), 6518-6548.
Juliano, R. L., & Ming, X. (2020). Carriers and Vehicles for Oligonucleotide Therapeutics. Molecular Therapy-Nucleic Acids, 19, 1338-1347.
Khan, I., Steeghs, K., & Ngondo, R. P. (2021). Antisense Oligonucleotide Therapeutics: Focus on Gene Splicing. Journal of Controlled Release, 329, 476-495.
Watts, J. K., Deleavey, G. F., Damha, M. J., & Vasquez, G. (2018). Anthraquinone Conjugates of 2′-O-Methyl Oligonucleotides for Duplex and Triplex RNA Targeting. Nucleic Acids Research, 46(11), 5686-5696.
Yuva-Aydemir, Y., Almeida, S., Krishnan, G., Gendron, T. F., and Gao, F-B. (2019). Transcription elongation factor AFF2/FMR2 regulates expression of expanded GGGGCC repeat-containing C90RF72 allele in ALS/FTD. Nat Commun. 10, 5466.
Lopez-Gonzalez, R., Yang, D., Pribadi, M., Kim, T.S., Krishnan, G., Choi, S.Y., Lee, S., Coppola, G., and Gao, F-B. (2019). Partial inhibition of the overactivated Ku80-dependent DNA repair pathway rescues neurodegeneration in C90RF72-ALS/FTD. PNAS 116, 9628-9633.
Capraro, V., Zane, L., Poncet, D., Perol, D., Galia, P., Preudhomme, C., Bonnefoy-Berard, N., Gilson, E., Thomas, X., El-Hamri, M., Chelghoun, Y., Michallet, M., Wattel, E., Mortreux, F., & Sibon, D. (2011). Telomere deregulations possess cytogenetic, phenotype, and prognostic specificities in acute leukemias. Experimental hematology, 39(2), 195-202.e2.

1. An oligonucleotide comprising a 5′ end, a 3′ end, and complementarity to a target polynucleotide, wherein the oligonucleotide comprises a poly-G sequence linked to the 5′ end and/or the 3′ end of the oligonucleotide, and wherein the poly-G sequence lacks complementarity to the target polynucleotide.

2. The oligonucleotide of claim 1, wherein the poly-G sequence comprises 2-30 G nucleotides.

3-9. (canceled)

10. The oligonucleotide of claim 1, wherein the poly-G sequence comprises G nucleotides that are consecutive, not consecutive, or a combination thereof.

11. The oligonucleotide of claim 1, wherein the poly-G sequence comprises or consists of:
(dG)(dG)(dG)(dG)(dG),
(dG)(dG)(dG)(dG)(dG)(dG),
(dG)(dG)(dG)(dG)(dG)(dG)(dG),
(dG)(dG)(dG)(dG)(dG)(dG)(dG)(dG),
(dG)(dG)(dG)(dG)(dG)(dG)(dG)(dG)(dG),
(SEQ ID NO: 56)
(dG)(dG)(dG)(dG)(dG)(dG)(dG)(dG)(dG)(dG),
(dG)(dG)(dG)(dN)(dG)(dG)(dG)(dN)(dG),
or
(SEQ ID NO: 58)
(dG)(dG)(dG)(dN)(dG)(dG)(G)(dN)(dG)(dG),
wherein each dN individually corresponds to a deoxyribonucleotide of dA, dT, or dC.

12-21. (canceled)

22. The oligonucleotide of claim 1, wherein the oligonucleotide and/or the poly-G sequence comprise one or more modified nucleotides, optionally wherein the one or more modified nucleotides each independently comprise a modification of a ribose group, a phosphate group, a nucleobase, or a combination thereof.

23-39. (canceled)

40. The oligonucleotide of claim 1, wherein a functional moiety is linked to the 5′ end or the 3′ end of the oligonucleotide.

41-42. (canceled)

43. The oligonucleotide of claim 1, comprising a formula:
A-B-C, wherein:
A comprises from about 0 to about 8 modified nucleotides;
B comprises from about 6 to about 18 deoxyribonucleic acid (DNA) nucleotides and/or DNA-like nucleotides; and
C comprises from about 0 to about 8 modified nucleotides,
wherein the overall length of the antisense oligonucleotide is about 10 to about 30 nucleotides.

44-47. (canceled)

48. The oligonucleotide of claim 1, comprising a nucleic acid sequence with at least 90% sequence identity to any one of the nucleic acid sequences of Table 1.

49. The oligonucleotide of claim 1, comprising:
a sequence modification pattern of
XsXsXsXsXsXsXsXsXsXsXsXsXsXsXsXsXsXsXsX, wherein:
s represents a phosphorothioate internucleoside linkage;
X comprises an adenosine, a guanosine, a cytidine, a thymine, or a uracil, wherein X comprises a 2′-O-(2-methoxyethyl) modification; and X comprises an adenosine, a guanosine, a cytidine, a thymine, or a uracil, wherein X comprises a 2′-deoxy modification; or
a sequence modification pattern of
XsXsXsXsXsXsXsXsXsXsXsXsXsXsXsXsXsX wherein:
s represents a phosphorothioate internucleoside linkage; and
X comprises an adenosine, a guanosine, a cytidine, a thymine, or a uracil, wherein X comprises a 2′-O-(2-methoxyethyl) modification.

50. (canceled)

51. The oligonucleotide of claim 1, wherein the target polynucleotide is a mammalian or viral mRNA, optionally wherein the target polynucleotide is an intronic or exonic region of the mammalian or viral mRNA.

52. The oligonucleotide of claim 1, wherein the target polynucleotide is selected from the group consisting of a EXOC2 gene, a Ku80 gene, a Task1 gene, a SOD1 gene, and a PCSK9 gene.

53-54. (canceled)

55. The oligonucleotide of claim 1, comprising:
the sequence
(SEQ ID NO: 48)
(eC)#(eA)(eG)#(eG)(eA)#(dT)#(dA)#(d5C)#(dA)#
(dT)#(dT)#(dT)#(d5C)#(dT)#(dA)#(eC)(eA)#(eG)
(eC)#(eU)(polyG),
wherein (#) indicates phosphorothioate linkage, (e) indicates a 2′MOE modification, (d) indicates a deoxyribonucleotide, (dN) indicates a deoxyribonucleotide of A, T, or C, (d5C) indicates 5-methyl cytosine, and (polyG) indicates 2-30 G nucleotides; or
the sequence
(SEQ ID NO: 49)
(eC)#(eA)(eG)#(eG)(eA)#(dT)#(dA)#(d5C)#(dA)#
(dT)#(dT)#(dT)#(d5C)#(dT)#(dA)#(eC)(eA)#(eG)
(eC)#(eU)(dG)ₓ,
wherein (#) indicates phosphorothioate linkage, (e) indicates a 2′MOE modification, (d) indicates a deoxyribonucleotide, (d5C) indicates 5-methyl cytosine, and x indicates an integer between 5-10: or
the sequence
(SEQ ID NO: 50)
(eC)#(eA)(eG)#(eG)(eA)#(dT)#(dA)#(d5C)#(dA)#
(dT)#(dT)#(dT)#(d5C)#(dT)#(dA)#(eC)(eA)#(eG)
(eC)#(eU)(dGdGdGdN)ₓ,
wherein (#) indicates phosphorothioate linkage, (e) indicates a 2′MOE modification, (d) indicates a deoxyribonucleotide, (d5C) indicates 5-methyl cytosine, (dN) indicates a deoxyribonucleotide of A, T, or C, and x indicates an integer between 2-10.

56-58. (canceled)

59. The oligonucleotide of claim 1, wherein the oligonucleotide is selected from the group consisting of an antisense oligonucleotide (ASO), a gapmer, a siRNA, a miRNA, a shRNA, a CRISPR guide, a DNA, an antisense mixmer, a miRNA inhibitor, a splice-switching oligonucleotide (SSO), a phosphorodiamidate morpholino oligomer (PMO), and a peptide nucleic acid (PNA).

60. The oligonucleotide of claim 1, wherein the oligonucleotide is a double-stranded RNA (dsRNA).

61. The oligonucleotide of claim 60, wherein the dsRNA comprises:
an antisense strand comprising a 3′ end, a 5′ end, and complementary to a target, optionally wherein the antisense strand is about 10-35 nucleotides in length;
a sense strand comprising a 3′ end, a 5′ end, and complementary to at least a portion of the antisense strand, optionally wherein the sense strand is about 10-35 nucleotides in length; and
a poly-G sequence linked to the 5′ end and/or the 3′ end of the antisense strand and/or the sense strand; or
an antisense strand of
(SEQ ID NO: 40)
V(mA)#(fC)#(mA)(fA)(fA)(fA)(mG)(fC)(mA)(fA)
(mA)(mA)(mC)(fA)(mG)(fG)(mU)(fC)(mU)(mA)
(mG)#(mA)#(mA)
and a sense strand of
(SEQ ID NO: 39)
(mC)#(mU)#(mA)(mG)(mA)(mC)(fC)(mU)(fG)(mU)
(dT)(mU)(mU)(mG)(mC)(mU)(mU)(mU)(mU)(mG)
(mU)GalNac,
wherein (#) indicates a phosphorothioate linkage, (mN) indicates a 2′-OMe modification, (fN) indicates a 2′-Fluoro modification, V indicates 5′-Vinyl phosphate, and GalNac indicates a N-acetylgalactosamine (GalNAc) conjugate; or
an antisense strand of
(SEQ ID NO: 51)
V(mA)#(fC)#(mA)(fA)(fA)(fA)(mG)(fC)(mA)(fA)
(mA)(mA)(mC)(fA)(mG)(fG)(mU)(fC)(mU)(mA)
(mG)#(mA)#(mA)(polyG),
wherein (#) indicates a phosphorothioate linkage, (mN) indicates a 2′-OMe modification, (fN) indicates a 2′-Fluoro modification, V indicates 5′-Vinyl phosphate, and (polyG) indicates 2-30 G nucleotides; or
an antisense strand of
(SEQ ID NO: 52)
V(mA)#(fC)#(mA)(fA)(fA)(fA)(mG)(fC)(mA)(fA)
(mA)(mA)(mC)(fA)(mG)(fG)(mU)(fC)(mU)(mA)
(mG)#(mA)#(mA)(dG)ₓ,
wherein (#) indicates a phosphorothioate linkage, (mN) indicates a 2′-OMe modification, (fN) indicates a 2′-Fluoro modification, V indicates 5′-Vinyl phosphate, and x indicates an integer between 5-10.

62-65. (canceled)

66. A pharmaceutical composition for inhibiting the expression of a gene in an organism comprising the oligonucleotide of claim 1.

67. The pharmaceutical composition of claim 66, wherein the gene is selected from the group consisting of a EXOC2 gene, a Ku80 gene, and a Task1 gene, optionally wherein the oligonucleotide inhibits the expression of the EXOC2 gene, the Ku80 gene, or the Task1 gene by at least about 50% or about 80%.

68-69. (canceled)

70. A vector comprising a regulatory sequence operably linked to a nucleotide sequence that encodes the oligonucleotide of claim 1.

71. The vector of claim 70, wherein the oligonucleotide inhibits the expression of a gene by at least about 30%, about 50%, or about 80%.

72-74. (canceled)

75. A recombinant adeno-associated virus (rAAV) comprising the vector of claim 70 and an AAV capsid.

76. A cell comprising the vector or the rAAV of claim 75.

77. A method for inhibiting expression of a gene in a cell, the method comprising:
(a) introducing into the cell the oligonucleotide of claim 1; and
(b) maintaining the cell produced in step (a) for a time sufficient to obtain degradation of a mRNA transcript of the gene, thereby inhibiting expression of the gene in the cell.

78. The method of claim 77, wherein the poly-G sequence directs the degradation from a lysosome, optionally wherein the gene is selected from the group consisting of a EXOC2 gene, a Ku80 gene, and a Task1 gene, optionally wherein the oligonucleotide inhibits the expression of the EXOC2 gene, the Ku80 gene, or the Task1 gene by at least about 50% or about 80%.

79-82. (canceled)

83. A method of treating or managing a disease associated with a gene comprising administering to a patient in need of such treatment a therapeutically effective amount of the oligonucleotide of claim 1.

84. The method of claim 83, wherein the gene is selected from the group consisting of a EXOC2 gene, a Ku80 gene, and a Task1 gene, optionally wherein the oligonucleotide inhibits the expression of the EXOC2 gene, the Ku80 gene, or the Task1 gene by at least about 50% or about 80%.

85-86. (canceled)

87. A method for degrading a target polynucleotide in a lysosome of a cell, the method comprising introducing the oligonucleotide of claim 1 into the cell, and maintaining the cell for a time sufficient to degrade the target polynucleotide in the lysosome of the cell.

88. The method of claim 87, wherein the target polynucleotide is a mammalian or viral mRNA, optionally wherein the target polynucleotide is an intronic or exonic region of the mammalian or viral mRNA, optionally wherein the target polynucleotide is selected from the group consisting of a EXOC2 gene, a Ku80 gene, and a Task1 gene, optionally wherein the oligonucleotide inhibits the expression of the EXOC2 gene, the Ku80 gene, or the Task1 gene by at least about 50%.

89-90. (canceled)

91. A method of treating or managing amyotrophic lateral sclerosis (ALS) in a patient, the method comprising administering to the patient a therapeutically effective amount of the oligonucleotide of claim 1, wherein the target polynucleotide is a SOD1 gene.

92. A method of treating or managing primary hyperlipidemia in a patient, the method comprising administering to the patient a therapeutically effective amount of the dsRNA of claim 60, wherein the target polynucleotide is a PCSK9 gene.

93. (canceled)

94. A method of reducing low density lipoprotein cholesterol (LDL-C) in a patient, the method comprising administering to the patient a therapeutically effective amount of the dsRNA of claim 60, wherein the target polynucleotide is a PSCK9 gene.


SEQUENCE LISTING (59 sequences)

SEQ ID NO: 1 - DNA, 20 bp, synthetic construct
  source 1..20: mol_type = other DNA
  modified_base 1..20: mod_base = OTHER; note = Phosphorothioate linkage
  modified_base order(1..5,16..20): mod_base = OTHER; note = 2-Prime-O-methoxy-ethyl nucleotides
  misc_feature order(1..5,16..20): note = RNA
  misc_feature 6..15: note = DNA
gcgactatacgcgcaatatg

SEQ ID NO: 2 - DNA, 20 bp, synthetic construct
  source 1..20: mol_type = other DNA
  modified_base 1..20: mod_base = OTHER; note = Phosphorothioate linkage
  modified_base order(1..5,16..20): mod_base = OTHER; note = 2-Prime-O-methoxy-ethyl nucleotides
  misc_feature order(1..5,16..20): note = RNA
  misc_feature 6..15: note = DNA
ttttggcttgtcccactcga

SEQ ID NO: 3 - DNA, 30 bp, synthetic construct
  source 1..30: mol_type = other DNA
  modified_base 1..21: mod_base = OTHER; note = Phosphorothioate linkage
  modified_base 21..30: mod_base = OTHER; note = Phosphodiester linkage
  modified_base order(1..5,16..20): mod_base = OTHER; note = 2-Prime-O-methoxy-ethyl nucleotides
  misc_feature order(1..5,16..20): note = RNA
  misc_feature order(6..15,21..30): note = DNA
ttttggcttgtcccactcgagggggggggg

SEQ ID NO: 4 - DNA, 27 bp, synthetic construct
  source 1..27: mol_type = other DNA
  modified_base 1..27: mod_base = OTHER; note = Phosphorothioate linkage
  modified_base order(1..5,16..20): mod_base = OTHER; note = 2-Prime-O-methoxy-ethyl nucleotides
  misc_feature order(6..15,21..27): note = DNA
  misc_feature order(1..5,16..20): note = RNA
ttttggcttgtcccactcgaggggggg

SEQ ID NO: 5 - DNA, 20 bp, synthetic construct
  source 1..20: mol_type = other DNA
  modified_base 1..20: mod_base = OTHER; note = Phosphodiester linkage
ttttggcttgtcccactcga

SEQ ID NO: 6 - DNA, 30 bp, synthetic construct; identical to SEQ ID NO: 3
  source 1..30: mol_type = other DNA
  modified_base 1..30: mod_base = OTHER; note = Phosphodiester linkage

SEQ ID NO: 7 - DNA, 34 bp, synthetic construct
  source 1..34: mol_type = other DNA
  modified_base 1..34: mod_base = OTHER; note = Phosphodiester linkage
gggggggttttggcttgtcccactcgaggggggg

SEQ ID NO: 8 - DNA, 20 bp, synthetic construct
  source 1..20: mol_type = other DNA
  modified_base 1..20: mod_base = OTHER; note = Phosphorothioate linkage
  modified_base order(1..5,16..20): mod_base = OTHER; note = 2-Prime-O-methoxy-ethyl nucleotides
  misc_feature 6..15: note = DNA
  misc_feature order(1..5,16..20): note = RNA
aagtgcatgcggctggtcta

SEQ ID NO: 9 - DNA, 25 bp, synthetic construct
  source 1..25: mol_type = other DNA
  modified_base 1..21: mod_base = OTHER; note = Phosphorothioate linkage
  modified_base 21..25: mod_base = OTHER; note = Phosphodiester linkage
  modified_base order(1..5,16..20): mod_base = OTHER; note = 2-Prime-O-methoxy-ethyl nucleotides
  misc_feature order(6..15,21..25): note = DNA
  misc_feature order(1..5,16..20): note = RNA
aagtgcatgcggctggtctaaaaaa

SEQ ID NO: 10 - DNA, 25 bp, synthetic construct
  source 1..25: mol_type = other DNA
  modified_base 1..21: mod_base = OTHER; note = Phosphorothioate linkage
  modified_base 21..25: mod_base = OTHER; note = Phosphodiester linkage
  modified_base order(1..5,16..20): mod_base = OTHER; note = 2-Prime-O-methoxy-ethyl nucleotides
  misc_feature order(1..5,16..20): note = RNA
  misc_feature order(6..15,21..25): note = DNA
aagtgcatgcggctggtctaccccc

SEQ ID NO: 11 - DNA, 25 bp, synthetic construct
  source 1..25: mol_type = other DNA
  modified_base 1..21: mod_base = OTHER; note = Phosphorothioate linkage
  modified_base 21..25: mod_base = OTHER; note = Phosphodiester linkage
  modified_base order(1..5,16..20): mod_base = OTHER; note = 2-Prime-O-methoxy-ethyl nucleotides
  misc_feature order(1..5,16..20): note = RNA
  misc_feature order(6..15,21..25): note = DNA
aagtgcatgcggctggtctaggggg

SEQ ID NO: 12 - DNA, 24 bp, synthetic construct
  source 1..24: mol_type = other DNA
  modified_base 1..21: mod_base = OTHER; note = Phosphorothioate linkage
  modified_base 21..24: mod_base = OTHER; note = Phosphodiester linkage
  modified_base order(1..5,16..20): mod_base = OTHER; note = 2-Prime-O-methoxy-ethyl nucleotides
  misc_feature order(1..5,16..20): note = RNA
  misc_feature order(6..15,21..24): note = DNA
aagtgcatgcggctggtctagggg

SEQ ID NO: 13 - DNA, 30 bp, synthetic construct
  source 1..30: mol_type = other DNA
  modified_base 1..21: mod_base = OTHER; note = Phosphorothioate linkage
  modified_base 21..30: mod_base = OTHER; note = Phosphodiester linkage
  modified_base order(1..5,16..20): mod_base = OTHER; note = 2-Prime-O-methoxy-ethyl nucleotides
  misc_feature order(1..5,16..20): note = RNA
  modified_base order(6..15,21..25): mod_base = OTHER; note = DNA
aagtgcatgcggctggtctagggggggggg

SEQ ID NO: 14 - DNA, 32 bp, synthetic construct
  source 1..32: mol_type = other DNA
  modified_base 1..21: mod_base = OTHER; note = Phosphorothioate linkage
  modified_base 21..32: mod_base = OTHER; note = Phosphodiester linkage
  modified_base order(1..5,16..20): mod_base = OTHER; note = 2-Prime-O-methoxy-ethyl nucleotides
  misc_feature order(1..5,16..20): note = RNA
  misc_feature order(6..15,21..25): note = DNA
aagtgcatgcggctggtctagggggggggggg

SEQ ID NO: 15 - RNA, 20 nt, synthetic construct
  source 1..20: mol_type = other RNA
  modified_base 1..20: mod_base = OTHER; note = 2-Prime-O-methoxy-ethyl nucleotides
  modified_base 1..20: mod_base = OTHER; note = Phosphorothioate linkage
aagtgcatgcggctggtcta

SEQ ID NO: 16 - DNA, 25 bp, synthetic construct
  source 1..25: mol_type = other DNA
  modified_base 1..21: mod_base = OTHER; note = Phosphorothioate linkage
  modified_base 21..25: mod_base = OTHER; note = Phosphodiester linkage
  modified_base order(1..5,16..20): mod_base = OTHER; note = 2-Prime-O-methoxy-ethyl nucleotides
  misc_feature 1..20: note = RNA
  misc_feature 21..25: note = DNA
aagtgcatgcggctggtctaggggg

SEQ ID NO: 17 - DNA, 20 bp, synthetic construct
  source 1..20: mol_type = other DNA
  modified_base 1..20: mod_base = OTHER; note = Phosphorothioate linkage
  modified_base order(1..5,16..20): mod_base = OTHER; note = 2-Prime-O-methoxy-ethyl nucleotides
  misc_feature order(1..5,16..20): note = RNA
  misc_feature 6..15: note = DNA
aagtgagtccgtccttaccc

SEQ ID NO: 18 - DNA, 30 bp, synthetic construct
  source 1..30: mol_type = other DNA
  modified_base 1..21: mod_base = OTHER; note = Phosphorothioate linkage
  modified_base 21..30: mod_base = OTHER; note = Phosphodiester linkage
  modified_base order(1..5,16..20): mod_base = OTHER; note = 2-Prime-O-methoxy-ethyl nucleotides
  misc_feature order(1..5,16..20): note = RNA
  misc_feature order(6..15,21..30): note = DNA
aagtgagtccgtccttacccgggggggggg

SEQ ID NO: 19 - DNA, 20 bp, synthetic construct
  source 1..20: mol_type = other DNA
  modified_base 1..20: mod_base = OTHER; note = Phosphorothioate linkage
  modified_base order(1..5,16..20): mod_base = OTHER; note = 2-Prime-O-methoxy-ethyl nucleotides
  misc_feature order(1..5,16..20): note = RNA
  misc_feature 6..15: note = DNA
attcaactgtgcctcggtgg

SEQ ID NO: 20 - DNA, 30 bp, synthetic construct
  source 1..30: mol_type = other DNA
  modified_base 1..21: mod_base = OTHER; note = Phosphorothioate linkage
  modified_base 21..30: mod_base = OTHER; note = Phosphodiester linkage
  modified_base order(1..5,16..20): mod_base = OTHER; note = 2-Prime-O-methoxy-ethyl nucleotides
  misc_feature order(1..5,16..20): note = RNA
  misc_feature order(6..15,21..30): note = DNA
attcaactgtgcctcggtgggggggggggg

SEQ ID NO: 21 - DNA, 20 bp, synthetic construct
  source 1..20: mol_type = other DNA
  modified_base 1..20: mod_base = OTHER; note = Phosphorothioate linkage
  modified_base order(1..5,16..20): mod_base = OTHER; note = 2-Prime-O-methoxy-ethyl nucleotides
  misc_feature order(1..5,16..20): note = RNA
  misc_feature 6..15: note = DNA
aagtgagtccgtccttaccc

SEQ ID NO: 22 - DNA, 30 bp, synthetic construct; identical to SEQ ID NO: 18
  source 1..30: mol_type = other DNA
  modified_base 1..21: mod_base = OTHER; note = Phosphorothioate linkage
  modified_base 21..30: mod_base = OTHER; note = Phosphodiester linkage
  modified_base order(1..5,16..20): mod_base = OTHER; note = 2-Prime-O-methoxy-ethyl nucleotides
  misc_feature order(1..5,16..20): note = RNA
  misc_feature order(6..15,21..30): note = DNA

SEQ ID NO: 23 - DNA, 20 bp, synthetic construct
  source 1..20: mol_type = other DNA
  modified_base 1..20: mod_base = OTHER; note = Phosphorothioate linkage
  modified_base order(1..5,16..20): mod_base = OTHER; note = 2-Prime-O-methoxy-ethyl nucleotides
  misc_feature order(1..5,16..20): note = RNA
  misc_feature 6..15: map = DNA
gaaggtccagtgctcgtagt

SEQ ID NO: 24 - DNA, 30 bp, synthetic construct
  source 1..30: mol_type = other DNA
  modified_base 1..21: mod_base = OTHER; note = Phosphorothioate linkage
  modified_base 21..30: mod_base = OTHER; note = Phosphodiester linkage
  modified_base order(1..5,16..20): mod_base = OTHER; note = 2-Prime-O-methoxy-ethyl nucleotides
  misc_feature order(1..5,16..20): note = RNA
  misc_feature order(6..15,21..30): note = DNA
gaaggtccagtgctcgtagtgggggggggg

SEQ ID NO: 25 - DNA, 20 bp, synthetic construct
  source 1..20: mol_type = other DNA
  modified_base 1..20: mod_base = OTHER; note = Phosphorothioate linkage
  modified_base order(1..5,16..20): mod_base = OTHER; note = 2-Prime-O-methoxy-ethyl nucleotides
  misc_feature order(1..5,16..20): note = RNA
  misc_feature 6..15: note = DNA
tgatgctcaccccgagtaac

SEQ ID NO: 26 - DNA, 30 bp, synthetic construct
  source 1..30: mol_type = other DNA
  modified_base 1..21: mod_base = OTHER; note = Phosphorothioate linkage
  modified_base 21..30: mod_base = OTHER; note = Phosphodiester linkage
  modified_base order(1..5,16..20): mod_base = OTHER; note = 2-Prime-O-methoxy-ethyl nucleotides
  misc_feature order(1..5,16..20): note = RNA
  misc_feature order(6..15,21..30): note = DNA
tgatgctcaccccgagtaacgggggggggg

SEQ ID NO: 27 - DNA, 20 bp, synthetic construct
  source 1..20: mol_type = other DNA
actgaagggggttctggagt

SEQ ID NO: 28 - DNA, 20 bp, synthetic construct
  source 1..20: mol_type = other DNA
ccaaacaagtcaggggaaga

SEQ ID NO: 29 - DNA, 19 bp, synthetic construct
  source 1..19: mol_type = other DNA
tgccatcgccaaggagtag

SEQ ID NO: 30 - DNA, 20 bp, synthetic construct
  source 1..20: mol_type = other DNA
tgcacagacggtcactcaaa

SEQ ID NO: 31 - DNA, 20 bp, synthetic construct
  source 1..20: mol_type = other DNA
ccccaattcagcagcatatt

SEQ ID NO: 32 - DNA, 20 bp, synthetic construct
  source 1..20: mol_type = other DNA
ccttcagccagactggagac

SEQ ID NO: 33 - DNA, 20 bp, synthetic construct
  source 1..20: mol_type = other DNA
ggtgctcatcggcttcttct

SEQ ID NO: 34 - DNA, 20 bp, synthetic construct
  source 1..20: mol_type = other DNA
gaagctgaaggccacgtact

SEQ ID NO: 35 - DNA, 20 bp, synthetic construct
  source 1..20: mol_type = other DNA
  modified_base order(1..5,16..20): mod_base = OTHER; note = 2-Prime-O-methoxy-ethyl nucleotides
  modified_base 1..20: mod_base = OTHER; note = Phosphorothioate linkage
  misc_feature order(1..5,16..20): note = RNA
  misc_feature 6..15: note = DNA
gcgactatacgcgcaatatg

SEQ ID NO: 36 - DNA, 20 bp, synthetic construct
  source 1..20: mol_type = other DNA
  modified_base order(1..5,16..20): mod_base = OTHER; note = 2-Prime-O-methoxy-ethyl nucleotides
  modified_base order(8,13): mod_base = OTHER; note = 5-methyl cytosine
  modified_base order(1..2,3..4,5..16,17..18,19..20): mod_base = OTHER; note = Phosphorothioate linkage
  misc_feature order(1..5,16..20): note = RNA
  misc_feature order(6..7,9..12,14..15): note = DNA
caggatacatttctacagct

SEQ ID NO: 37 - DNA, 30 bp, synthetic construct
  source 1..30: mol_type = other DNA
  modified_base order(1..5,16..20): mod_base = OTHER; note = 2-Prime-O-methoxy-ethyl nucleotides
  modified_base order(8,13): mod_base = OTHER; note = 5-methyl cytosine
  modified_base order(1..2,3..4,5..16,17..18,19..20): mod_base = OTHER; note = Phosphorothioate linkage
  misc_feature order(1..5,16..20): note = RNA
  misc_feature order(6..7,9..12,14..15,21..30): note = DNA
caggatacatttctacagctgggggggggg

SEQ ID NO: 38 - DNA, 29 bp, synthetic construct
  source 1..29: mol_type = other DNA
  modified_base order(1..5,16..20): mod_base = OTHER; note = 2-Prime-O-methoxy-ethyl nucleotides
  modified_base order(8,13): mod_base = OTHER; note = 5-methyl cytosine
  modified_base order(1..2,3..4,5..16,17..18,19..20): mod_base = OTHER; note = Phosphorothioate linkage
  misc_feature order(6..7,9..12,14..15,21..29): note = DNA
  misc_feature order(1..5,16..20): note = RNA
caggatacatttctacagctgggagggag

SEQ ID NO: 39 - RNA, 21 nt, synthetic construct
  source 1..21: mol_type = other RNA
  modified_base order(1..6,8,10,12..21): mod_base = OTHER; note = 2-Prime-O-methyl nucleotides
  modified_base order(7,9): mod_base = OTHER; note = 2-Prime-fluoro nucleotides
  modified_base 11: mod_base = OTHER; note = Thymine
  modified_base 1..3: mod_base = OTHER; note = Phosphorothioate linkage
ctagacctgttttgcttttgt

SEQ ID NO: 40 - RNA, 23 nt, synthetic construct
  source 1..23: mol_type = other RNA
  modified_base order(1,3,7,9,11..13,15,17,19..23): mod_base = OTHER; note = 2-Prime-O-methyl nucleotides
  modified_base order(2,4..6,8,10,14,16,18): mod_base = OTHER; note = 2-Prime-fluoro nucleotides
  modified_base order(1..3,21..23): mod_base = OTHER; note = Phosphorothioate linkage
acaaaagcaaaacaggtctagaa

SEQ ID NO: 41 - RNA, 28 nt, synthetic construct
  source 1..28: mol_type = other RNA
  modified_base order(1,3,7,9,11..13,15,17,19..23): mod_base = OTHER; note = 2-Prime-O-methyl nucleotides
  modified_base order(2,4..6,8,10,14,16,18): mod_base = OTHER; note = 2-Prime-fluoro nucleotides
  modified_base 24..28: mod_base = OTHER; note = 2-Prime-deoxyribonucleosides
  modified_base order(1..3,21..23): mod_base = OTHER; note = Phosphorothioate linkage
acaaaagcaaaacaggtctagaaggggg

SEQ ID NO: 42 - DNA, 21 bp, synthetic construct
  source 1..21: mol_type = other DNA
actggtggtccatgaaaaagc

SEQ ID NO: 43 - DNA, 20 bp, synthetic construct
  source 1..20: mol_type = other DNA
aacgacttccagcgtttcct

SEQ ID NO: 44 - DNA, 20 bp, synthetic construct
  source 1..20: mol_type = other DNA
ccttcttcctggcttcctgg

SEQ ID NO: 45 - DNA, 20 bp, synthetic construct
  source 1..20: mol_type = other DNA
tcacaaaagccgaggtagcc

SEQ ID NO: 46 - DNA, 18 bp, synthetic construct
  source 1..18: mol_type = other DNA
tgcaccaccaactgctta

SEQ ID NO: 47 - DNA, 19 bp, synthetic construct
  source 1..19: mol_type = other DNA
ggatgcagggatgatgttc

SEQ ID NO: 48 - DNA, 22 bp, synthetic construct
  source 1..22: mol_type = other DNA
  modified_base order(1..5,16..20): mod_base = OTHER; note = 2-Prime-O-methoxy-ethyl nucleotides
  modified_base order(8,13): mod_base = OTHER; note = 5-methyl cytosine
  modified_base order(1..2,3..4,5..16,17..18,19..20): mod_base = OTHER; note = Phosphorothioate linkage
  variation 22: note = G residue can be repeated up to twenty-eight times.
  misc_feature order(1..5,16..20): note = RNA
  misc_feature order(6..7,9..12,14..15,21..22): note = DNA
caggatacatttctacagctgg

SEQ ID NO: 49 - DNA, 25 bp, synthetic construct
  source 1..25: mol_type = other DNA
  modified_base order(1..5,16..20): mod_base = OTHER; note = 2-Prime-O-methoxy-ethyl nucleotides
  modified_base order(8,13): mod_base = OTHER; note = 5-methyl cytosine
  modified_base order(1..2,3..4,5..16,17..18,19..20): mod_base = OTHER; note = Phosphorothioate linkage
  variation 25: note = G residue can be repeated up to five times.
  misc_feature order(1..5,16..20): note = RNA
  misc_feature order(6..7,9..12,14..15,21..25): note = DNA
caggatacatttctacagctggggg

SEQ ID NO: 50 - DNA, 28 bp, synthetic construct
  source 1..28: mol_type = other DNA
  modified_base order(1..5,16..20): mod_base = OTHER; note = 2-Prime-O-methoxy-ethyl nucleotides
  modified_base order(8,13): mod_base = OTHER; note = 5-methyl cytosine
  modified_base order(1..2,3..4,5..16,17..18,19..20): mod_base = OTHER; note = Phosphorothioate linkage
  variation 25..28: note = GGGH residues can be repeated up to eight times.
  misc_feature order(1..5,16..20): note = RNA
  misc_feature order(6..7,9..12,14..15,21..28): note = DNA
caggatacatttctacagctggghgggh

SEQ ID NO: 51 - RNA, 25 nt, synthetic construct
  source 1..25: mol_type = other RNA
  modified_base order(1,3,7,9,11..13,15,17,19..23): mod_base = OTHER; note = 2-Prime-O-methyl nucleotides
  modified_base order(2,4..6,8,10,14,16,18): mod_base = OTHER; note = 2-Prime-fluoro nucleotides
  modified_base order(1..3,21..23): mod_base = OTHER; note = Phosphorothioate linkage
  variation 24: note = G residue can be repeated up to twenty-eight times.
acaaaagcaaaacaggtctagaagg

SEQ ID NO: 52 - RNA, 28 nt, synthetic construct
  source 1..28: mol_type = other RNA
  modified_base order(1,3,7,9,11..13,15,17,19..23): mod_base = OTHER; note = 2-Prime-O-methyl nucleotides
  modified_base order(2,4..6,8,10,14,16,18): mod_base = OTHER; note = 2-Prime-fluoro nucleotides
  modified_base order(1..3,21..23): mod_base = OTHER; note = Phosphorothioate linkage
  variation 28: note = G residue can be repeated up to five times.
acaaaagcaaaacaggtctagaaggggg

SEQ ID NO: 53 - DNA, 18 bp, synthetic construct
  source 1..18: mol_type = other DNA
  modified_base 1..18: mod_base = OTHER; note = Phosphorothioate internucleoside linkage
  modified_base order(1..5,15..18): mod_base = OTHER; note = 2-Prime-O-(2-methoxyethyl) nucleotides
  misc_feature order(1..5,15..18): note = RNA
  misc_feature 6..14: note = DNA
gcaatacatggattgggg

SEQ ID NO: 54 - RNA, 18 nt, synthetic construct
  source 1..18: mol_type = other RNA
  modified_base 1..18: mod_base = OTHER; note = Phosphorothioate internucleoside linkage
  modified_base 1..18: mod_base = OTHER; note = 2-Prime-O-(2-methoxyethyl) nucleotides
gcaatacatggattgggg

SEQ ID NO: 55 - RNA, 18 nt, synthetic construct
  source 1..18: mol_type = other RNA
  modified_base 1..18: mod_base = OTHER; note = Phosphorothioate internucleoside linkage
  modified_base 1..18: mod_base = OTHER; note = 2-Prime-O-(2-methoxyethyl) nucleotides
gtacaaactccggagagc

SEQ ID NO: 56 - DNA, 10 bp, synthetic construct
  source 1..10: mol_type = other DNA
gggggggggg

SEQ ID NO: 57 - DNA, 10 bp, synthetic construct
  source 1..10: mol_type = other DNA
  variation 1: note = G residue can be repeated upto twenty times.
gggggggggg

SEQ ID NO: 58 - DNA, 10 bp, synthetic construct
  source 1..10: mol_type = other DNA
ggghggghgg

SEQ ID NO: 59 - DNA, 12 bp, synthetic construct
  source 1..12: mol_type = other DNA
gggggggggggg